(19) United States
(12) Patent Application Publication
(10) Pub. No.: US 2026/0158222 A1
(43) Pub. Date: Jun. 11, 2026

(54) Syringe Detect Force Mechanism

(71) Applicant: Becton, Dickinson and Company, Franklin Lakes, NJ (US)

(72) Inventors: Hong Zhu, Glen Rock, NJ (US); Gary Ellerbusch, Nutley, NJ (US); John Picot, Landing, NJ (US)

(21) Appl. No.: 18/969,813

(22) Filed: Dec. 5, 2024

(51) Int. Cl. A61M 5/31

(52) U.S. Cl. CPC A61M 5/31; A61M 2205/3331

(57) ABSTRACT

A flow sensor system may include a flow sensor and a base configured to connect to the flow sensor. The flow sensor may include a flow sensor housing; a flow tube including a first end and a second end; a fluid injection port between the first end and the second end; and at least one sensor configured to characterize at least one attribute of a fluid in the flow tube. The base may include a base housing; at least one force sensor configured to generate at least one force signal indicative of at least one force applied to the base housing; and at least one processor configured to determine, based on the at least one force signal, a connection of a medical device to the fluid injection port of the flow sensor or a disconnection of the medical device from the fluid injection port of the flow sensor.

### BACKGROUND

### 1. Technical Field

This disclosure relates generally to flow sensors and, in some non-limiting embodiments or aspects, to a mechanism for detecting a connection of a medical device to a flow sensor.

### 2. Technical Considerations

A flow sensor system may be used to provide a record of, and electronically measure, bolus delivery to a patient. A flow sensor may include a needleless connector to which medication-filled medical devices (e.g., syringes, etc.) are connected for medication administration. As use of the Internet of Things (IoT) spreads in the medical field, there may be a need to automatically detect/record when a medical device (e.g., a syringe, etc.) is attached to or removed from a flow sensor.

### SUMMARY

According to some non-limiting embodiments or aspects, provided is a flow sensor system, including: a flow sensor including: a flow sensor housing; a flow tube including a fluid inlet at a first end of the flow tube, a fluid outlet at a second end of the flow tube opposite the first end of the flow tube, and a fluid injection port between the first end and the second end of the flow tube; and at least one sensor configured to characterize at least one attribute of a fluid in the flow tube; and a base configured to connect to the flow sensor, wherein the base includes: a base housing; at least one force sensor configured to generate at least one force signal indicative of at least one force applied to the base housing; and at least one processor configured to determine, based on the at least one force signal, a connection of a medical device to the fluid injection port of the flow sensor or a disconnection of the medical device from the fluid injection port of the flow sensor.

In some non-limiting embodiments or aspects, the at least one processor is further configured to determine, based on the at least one force signal, a connection of the flow sensor to the base or a disconnection of the flow sensor from the base.

In some non-limiting embodiments or aspects, the at least one force sensor includes a first force sensor configured to generate a first force signal indicative of the at least one force applied to the base housing and a second force sensor configured to generate a second force signal indicative of the at least one force applied to the base housing, and wherein the at least one force signal includes a difference between the first force signal and the second force signal that is indicative of at least one of the following: a tension force or a compression force applied to the base housing, a torsional force applied to the base housing, or any combination thereof.

In some non-limiting embodiments or aspects, the first force sensor and the second force sensor are mounted in-line with each other and parallel to a longitudinal axis of the fluid injection port, and wherein the difference between the first force signal and the second force signal is indicative of the tension force or the compression force applied to the base housing.

In some non-limiting embodiments or aspects, the first force sensor and the second force sensor are mounted in-line with each other and perpendicular to a longitudinal axis of the fluid injection port, and wherein the difference between the first force signal and the second force signal is indicative of the torsional force applied to the base housing.

In some non-limiting embodiments or aspects, the at least one force sensor further includes a third force sensor configured to generate a third force signal indicative of the at least one force applied to the base housing and a fourth force sensor configured to generate a fourth force signal indicative of the at least one force applied to the base housing, wherein the third force sensor and the fourth force sensor are mounted in-line with each other and perpendicular to a longitudinal axis of the fluid injection port and parallel to the first force sensor and the second force sensor mounted in-line with each other, and wherein the at least one force signal includes a difference between the first force signal or the second force signal and the third force signal or the fourth force signal that is indicative of at least one of the following: a tension force or a compression force applied to the base housing, a torsional force applied to the base housing, or any combination thereof.

In some non-limiting embodiments or aspects, the first force sensor and the second force sensor are offset from each other along each of a longitudinal axis of the fluid injection port and a lateral axis of the fluid injection port, and wherein the difference between the first force signal and the second force signal is indicative of the combination of the tension force or the compression force applied to the base housing and the torsional force applied to the base housing.

In some non-limiting embodiments or aspects, the base further includes: a printed circuit board (PCB), wherein the at least one force sensor is mounted on the PCB.

In some non-limiting embodiments or aspects, the at least one force sensor is in direct physical contact with an inner surface of base housing.

In some non-limiting embodiments or aspects, the base further includes: at least one actuator arm in physical contact with the at least one force sensor and an inner surface of the base housing, wherein the at least one actuator arm is configured to transfer at least a portion of the at least one force applied to the base housing to the at least one force sensor.

In some non-limiting embodiments or aspects, the flow sensor housing extends between a first end and a second end opposite the first end, wherein the fluid injection port extends from the flow sensor housing at the second end of the flow sensor housing, wherein the flow sensor housing includes a first arced flange that at least partially surrounds the fluid injection port, wherein the base housing extends between a first end and a second end opposite the first end and between a first side and a second side opposite the first side, wherein the first side of the base housing includes an opening that extends between the first end and the second end opposite the first end, wherein the opening is configured to receive the flow sensor housing, wherein the opening extends from the first side at the second end of the base housing toward the second side of the base housing as an at least partially open bore, wherein the at least partially open bore is partially surrounded by a second arced flange, wherein the second arced flange is configured to receive and partially surround the fluid injection port with the first arced flange of the flow sensor housing between the second arced flange of the base housing and the second side of the base housing, and wherein the at least one force sensor is configured to receive the at least one force applied to the base housing via the second arced flange of the base housing to generate the at least one force signal indicative of the at least one force applied to the base housing.

In some non-limiting embodiments or aspects, the first arced flange of the flow sensor housing includes a first face that faces the flow sensor housing and a second face opposite the first face, wherein the second arced flange of the base housing includes a first face that faces the first side of the base housing and away from the second side of the base housing and a second face opposite the first face that faces toward the second side of the base housing, wherein, when the fluid injection port is received within and partially surrounded by the second arced flange of base housing, the first arced flange of the flow sensor housing is rotatable with respect to the second arced flange of the base housing to bring the first face of the first arced flange flow of the flow sensor housing into contact with the first face of the second arced flange of the base housing, and wherein the at least one force sensor is configured to receive the at least one force applied to the base housing via the first face of the second arced flange of the base housing to generate the at least one force signal indicative of the at least one force applied to the base housing.

In some non-limiting embodiments or aspects, the flow sensor further includes a flow sensor electrical contact in electrical communication with the at least one sensor, wherein the base further includes a base electrical contact in electrical communication with the one or more processors, and wherein the flow sensor electrical contact is in electrical communication with the base electrical contact when the flow sensor is connected to the base.

In some non-limiting embodiments or aspects, the fluid injection port includes a threaded connector, and wherein the medical device includes a syringe including a complementary threaded connector configured to complementarily mate with the threaded connector of the fluid injection port.

According to some non-limiting embodiments or aspects, provided is a base for a flow sensor, including: a base housing; at least one force sensor configured to generate at least one force signal indicative of at least one force applied to the base housing; and at least one processor configured to determine, based on the at least one force signal, a connection of a medical device to a fluid injection port of a flow sensor connected to the base or a disconnection of the medical device from the fluid injection port of the flow sensor connected to the base.

In some non-limiting embodiments or aspects, the at least one processor is further configured to determine, based on the at least one force signal, a connection of the flow sensor to the base or a disconnection of the flow sensor from the base.

In some non-limiting embodiments or aspects, the at least one force sensor includes a first force sensor configured to generate a first force signal indicative of the at least one force applied to the base housing and a second force sensor configured to generate a second force signal indicative of the at least one force applied to the base housing, and wherein the at least one force signal includes a difference between the first force signal and the second force signal that is indicative of at least one of the following: a tension force or a compression force applied to the base housing, a torsional force applied to the base housing, or any combination thereof.

In some non-limiting embodiments or aspects, the base further includes: a printed circuit board (PCB), wherein the at least one force sensor is mounted on the PCB.

In some non-limiting embodiments or aspects, the base further includes: at least one actuator arm in physical contact with the at least one force sensor and an inner surface of the base housing, wherein the at least one actuator arm is configured to transfer at least a portion of the at least one force applied to the base housing to the at least one force sensor.

According to some non-limiting embodiments or aspects, provided is a system, including: a fluid injection port; a housing connected to the fluid injection port; at least one force sensor configured to generate at least one force signal indicative of at least one force applied to the housing; and at least one processor configured to determine, based on the at least one force signal, a connection of a medical device to the fluid injection port or a disconnection of the medical device from the fluid injection port.

Further, some non-limiting embodiments or aspects are set forth in the following numbered clauses:
Clause 1: A flow sensor system, comprising: a flow sensor including: a flow sensor housing; a flow tube including a fluid inlet at a first end of the flow tube, a fluid outlet at a second end of the flow tube opposite the first end of the flow tube, and a fluid injection port between the first end and the second end of the flow tube; and at least one sensor configured to characterize at least one attribute of a fluid in the flow tube; and a base configured to connect to the flow sensor, wherein the base includes: a base housing; at least one force sensor configured to generate at least one force signal indicative of at least one force applied to the base housing; and at least one processor configured to determine, based on the at least one force signal, a connection of a medical device to the fluid injection port of the flow sensor or a disconnection of the medical device from the fluid injection port of the flow sensor.
Clause 2: The flow sensor system of clause 1, wherein the at least one processor is further configured to determine, based on the at least one force signal, a connection of the flow sensor to the base or a disconnection of the flow sensor from the base.
Clause 3: The flow sensor system of clause 1 or clause 2, wherein the at least one force sensor includes a first force sensor configured to generate a first force signal indicative of the at least one force applied to the base housing and a second force sensor configured to generate a second force signal indicative of the at least one force applied to the base housing, and wherein the at least one force signal includes a difference between the first force signal and the second force signal that is indicative of at least one of the following: a tension force or a compression force applied to the base housing, a torsional force applied to the base housing, or any combination thereof.
Clause 4: The flow sensor system of any of clauses 1-3, wherein the first force sensor and the second force sensor are mounted in-line with each other and parallel to a longitudinal axis of the fluid injection port, and wherein the difference between the first force signal and the second force signal is indicative of the tension force or the compression force applied to the base housing.
Clause 5: The flow sensor system of any of clauses 1-4, wherein the first force sensor and the second force sensor are mounted in-line with each other and perpendicular to a longitudinal axis of the fluid injection port, and wherein the difference between the first force signal and the second force signal is indicative of the torsional force applied to the base housing.
Clause 6: The flow sensor system of any of clauses 1-5, wherein the at least one force sensor further includes a third force sensor configured to generate a third force signal indicative of the at least one force applied to the base housing and a fourth force sensor configured to generate a fourth force signal indicative of the at least one force applied to the base housing, wherein the third force sensor and the fourth force sensor are mounted in-line with each other and perpendicular to a longitudinal axis of the fluid injection port and parallel to the first force sensor and the second force sensor mounted in-line with each other, and wherein the at least one force signal includes a difference between the first force signal or the second force signal and the third force signal or the fourth force signal that is indicative of at least one of the following: a tension force or a compression force applied to the base housing, a torsional force applied to the base housing, or any combination thereof.
Clause 7: The flow sensor system of any of clauses 1-6, wherein the first force sensor and the second force sensor are offset from each other along each of a longitudinal axis of the fluid injection port and a lateral axis of the fluid injection port, and wherein the difference between the first force signal and the second force signal is indicative of the combination of the tension force or the compression force applied to the base housing and the torsional force applied to the base housing.
Clause 8: The flow sensor system of any of clauses 1-7, wherein the base further includes: a printed circuit board (PCB), wherein the at least one force sensor is mounted on the PCB.
Clause 9: The flow sensor system of any of clauses 1-8, wherein the at least one force sensor is in direct physical contact with an inner surface of base housing.
Clause 10: The flow sensor system of any of clauses 1-9, wherein the base further includes: at least one actuator arm in physical contact with the at least one force sensor and an inner surface of the base housing, wherein the at least one actuator arm is configured to transfer at least a portion of the at least one force applied to the base housing to the at least one force sensor.
Clause 11: The flow sensor system of any of clauses 1-10, wherein the flow sensor housing extends between a first end and a second end opposite the first end, wherein the fluid injection port extends from the flow sensor housing at the second end of the flow sensor housing, wherein the flow sensor housing includes a first arced flange that at least partially surrounds the fluid injection port, wherein the base housing extends between a first end and a second end opposite the first end and between a first side and a second side opposite the first side, wherein the first side of the base housing includes an opening that extends between the first end and the second end opposite the first end, wherein the opening is configured to receive the flow sensor housing, wherein the opening extends from the first side at the second end of the base housing toward the second side of the base housing as an at least partially open bore, wherein the at least partially open bore is partially surrounded by a second arced flange, wherein the second arced flange is configured to receive and partially surround the fluid injection port with the first arced flange of the flow sensor housing between the second arced flange of the base housing and the second side of the base housing, and wherein the at least one force sensor is configured to receive the at least one force applied to the base housing via the second arced flange of the base housing to generate the at least one force signal indicative of the at least one force applied to the base housing.
Clause 12: The flow sensor system of any of clauses 1-11, wherein the first arced flange of the flow sensor housing includes a first face that faces the flow sensor housing and a second face opposite the first face, wherein the second arced flange of the base housing includes a first face that faces the first side of the base housing and away from the second side of the base housing and a second face opposite the first face that faces toward the second side of the base housing, wherein, when the fluid injection port is received within and partially surrounded by the second arced flange of base housing, the first arced flange of the flow sensor housing is rotatable with respect to the second arced flange of the base housing to bring the first face of the first arced flange flow of the flow sensor housing into contact with the first face of the second arced flange of the base housing, and wherein the at least one force sensor is configured to receive the at least one force applied to the base housing via the first face of the second arced flange of the base housing to generate the at least one force signal indicative of the at least one force applied to the base housing.
Clause 13: The flow sensor system of any of clauses 1-12, wherein the flow sensor further includes a flow sensor electrical contact in electrical communication with the at least one sensor, wherein the base further includes a base electrical contact in electrical communication with the one or more processors, and wherein the flow sensor electrical contact is in electrical communication with the base electrical contact when the flow sensor is connected to the base.
Clause 14: The flow sensor system of any of clauses 1-13, wherein the fluid injection port includes a threaded connector, and wherein the medical device includes a syringe including a complementary threaded connector configured to complementarily mate with the threaded connector of the fluid injection port.
Clause 15: A base for a flow sensor, comprising: a base housing; at least one force sensor configured to generate at least one force signal indicative of at least one force applied to the base housing; and at least one processor configured to determine, based on the at least one force signal, a connection of a medical device to a fluid injection port of a flow sensor connected to the base or a disconnection of the medical device from the fluid injection port of the flow sensor connected to the base.
Clause 16: The base of clause 15 wherein the at least one processor is further configured to determine, based on the at least one force signal, a connection of the flow sensor to the base or a disconnection of the flow sensor from the base.
Clause 17: The base of clause 15 or clause 16, wherein the at least one force sensor includes a first force sensor configured to generate a first force signal indicative of the at least one force applied to the base housing and a second force sensor configured to generate a second force signal indicative of the at least one force applied to the base housing, and wherein the at least one force signal includes a difference between the first force signal and the second force signal that is indicative of at least one of the following: a tension force or a compression force applied to the base housing, a torsional force applied to the base housing, or any combination thereof.
Clause 18: The base of any of clauses 15-17, wherein the base further includes: a printed circuit board (PCB), wherein the at least one force sensor is mounted on the PCB.
Clause 19: The base of any of clauses 15-18, wherein the base further includes: at least one actuator arm in physical contact with the at least one force sensor and an inner surface of the base housing, wherein the at least one actuator arm is configured to transfer at least a portion of the at least one force applied to the base housing to the at least one force sensor.
Clause 20: A system, comprising: a fluid injection port; a housing connected to the fluid injection port; at least one force sensor configured to generate at least one force signal indicative of at least one force applied to the housing; and at least one processor configured to determine, based on the at least one force signal, a connection of a medical device to the fluid injection port or a disconnection of the medical device from the fluid injection port.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosed subject matter.

### BRIEF DESCRIPTION OF THE DRAWINGS

Additional advantages and details are explained in greater detail below with reference to the non-limiting, exemplary embodiments that are illustrated in the accompanying schematic figures, in which:

FIG. 1 is a diagram of some non-limiting embodiments or aspects of an environment in which systems, devices, products, apparatus, and/or methods, described herein, may be implemented;

FIG. 2 is a diagram of some non-limiting embodiments or aspects of components of one or more devices and/or one or more systems of FIG. 1;

FIGS. 3A-3E depict perspective views of a flow sensor system, according to some non-limiting embodiments or aspects;

FIG. 4A depicts a perspective view of a flow sensor of a flow sensor system, according to some non-limiting embodiments or aspects;

FIG. 4B depicts a side view of a flow sensor of a flow sensor system, according to some non-limiting embodiments or aspects;

FIG. 4C depicts a cutaway view of a flow sensor of a flow sensor system, according to some non-limiting embodiments or aspects;

FIGS. 5A and 5B depict perspective views of a base of a flow sensor system, according to some non-limiting embodiments or aspects;

FIG. 6A depicts a perspective view of positioning of a flow sensor relative to a base during connection of a flow sensor system, according to some non-limiting embodiments or aspects;

FIG. 6B depicts a cross-sectional perspective view of a flow sensor relative to a base during connection of a flow sensor system, according to some non-limiting embodiments or aspects;

FIG. 6C depicts a sequence of perspective views showing connection of a flow sensor to a base, according to some non-limiting embodiments or aspects;

FIG. 6D is a sequence of perspective views showing disconnection of a flow sensor from a base, according to some non-limiting embodiments or aspects;

FIG. 7 depicts a perspective view of some non-limiting embodiments or aspects of a connection between components of a flow sensor system;

FIG. 8 depicts a perspective view of some non-limiting embodiments or aspects of a connection between components of a flow sensor system;

FIG. 9 depicts a sectional view of view of some non-limiting embodiments or aspects of a connection between components of a flow sensor system;

FIG. 10 depicts a sectional view of view of some non-limiting embodiments or aspects of a connection between components of a flow sensor system;

FIG. 11 depicts a perspective view of some non-limiting embodiments or aspects of a connection between components of a flow sensor system;

FIG. 12 depicts a sectional view of some non-limiting embodiments or aspects of a connection between components of a flow sensor system; and

FIG. 13 depicts a cutaway view of a fluid injection port and components proximate thereof of a flow sensor system, according to some non-limiting embodiments or aspects;

FIG. 14 depicts a cross-sectional view of a base of a flow sensor system including a force sensor mounted in direct physical contact with an inner wall of a base housing, according to some non-limiting embodiments or aspects, according to some non-limiting embodiments or aspects;

FIG. 15 depicts a cross-sectional view of a flow sensor system including an actuator arm in physical contact with a force sensor and an inner wall of a base housing, according to some non-limiting embodiments or aspects;

FIG. 16 depicts a side view of a flow sensor of a flow sensor and a corresponding free body diagram thereof, according to some non-limiting embodiments or aspects;

FIGS. 17 and 18 depict perspective views of force applied to connect a medical device to a flow sensor system, according to some non-limiting embodiments or aspects;

FIG. 19 is a cross-sectional view of a medical device connected to a flow sensor system, according to some non-limiting embodiments or aspects;

FIG. 20A depicts an in-line arrangement of force sensors, according to some non-limiting embodiments or aspects;

FIG. 20B depicts a parallel and in-line arrangement of force sensors, according to some non-limiting embodiments or aspects; and

FIG. 20C depicts an offset arrangement of force sensors, according to some non-limiting embodiments or aspects.

### DETAILED DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the embodiments as they are oriented in the drawing figures. However, it is to be understood that the present disclosure may assume various alternative variations and step sequences, except where expressly specified to the contrary. It is also to be understood that the specific devices and processes illustrated in the attached drawings, and described in the following specification, are simply exemplary and some non-limiting embodiments or aspects of the disclosed subject matter. Hence, specific dimensions and other physical characteristics related to some non-limiting embodiments or aspects disclosed herein are not to be considered as limiting.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, and/or the like) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise. In addition, reference to an action being “based on” a condition may refer to the action being “in response to” the condition. For example, the phrases “based on” and “in response to” may, in some non-limiting embodiments or aspects, refer to a condition for automatically triggering an action (e.g., a specific operation of an electronic device, such as a computing device, a processor, and/or the like).

Some of the techniques described herein may be implemented by one or more computer programs executed by one or more processors residing, for example on a medical device, such as a flow sensor, or the like. The computer programs may include processor-executable instructions that are stored on a non-transitory tangible computer readable medium. The computer programs may also include stored data. Non-limiting examples of the non-transitory tangible computer readable medium are nonvolatile memory, magnetic storage, and optical storage.

As used herein, the term “communication” may refer to the reception, receipt, transmission, transfer, provision, and/or the like of data (e.g., information, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection (e.g., a direct communication connection, an indirect communication connection, and/or the like) that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit processes information received from the first unit and communicates the processed information to the second unit. In some non-limiting embodiments or aspects, a message may refer to a network packet (e.g., a data packet and/or the like) that includes data. It will be appreciated that numerous other arrangements are possible.

As used herein, the term “computing device” may refer to one or more electronic devices configured to process data. A computing device may, in some examples, include the necessary components to receive, process, and output data, such as a processor, a display, a memory, an input device, a network interface, and/or the like. A computing device may be a mobile device. As an example, a mobile device may include a cellular phone (e.g., a smartphone or standard cellular phone), a portable computer, a wearable device (e.g., watches, glasses, lenses, clothing, and/or the like), a personal digital assistant (PDA), and/or other like devices. A computing device may also be a desktop computer or other form of non-mobile computer.

As used herein, the term “server” may refer to or include one or more computing devices that are operated by or facilitate communication and processing for multiple parties in a network environment, such as the Internet, although it will be appreciated that communication may be facilitated over one or more public or private network environments and that various other arrangements are possible. Further, multiple computing devices (e.g., servers, point-of-sale (POS) devices, mobile devices, etc.) directly or indirectly communicating in the network environment may constitute a “system.”

As used herein, the term “system” may refer to one or more computing devices or combinations of computing devices (e.g., processors, servers, client devices, software applications, components of such, and/or the like). Reference to “a device,” “a server,” “a processor,” and/or the like, as used herein, may refer to a previously-recited device, server, or processor that is recited as performing a previous step or function, a different device, server, or processor, and/or a combination of devices, servers, and/or processors. For example, as used in the specification and the claims, a first device, a first server, or a first processor that is recited as performing a first step or a first function may refer to the same or different device, server, or processor recited as performing a second step or a second function.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, inserting and the like via a hardware element.

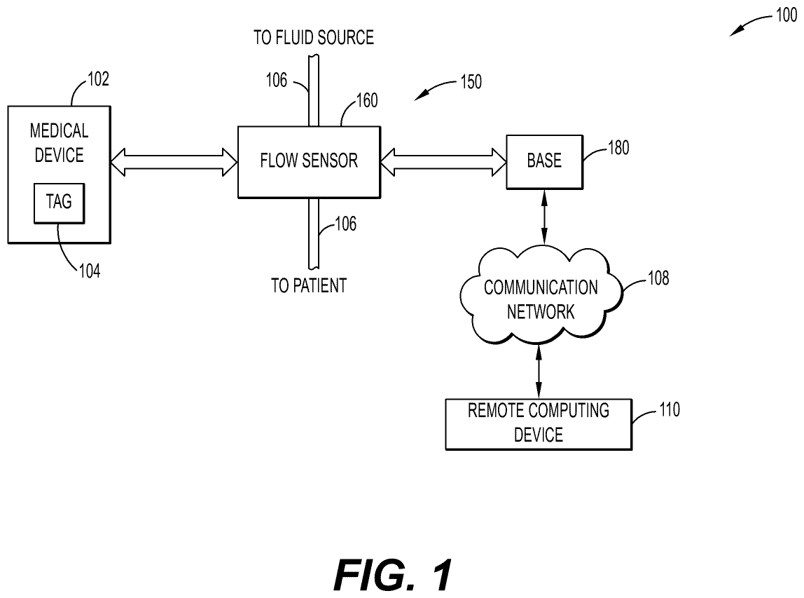

Referring now to FIG. 1, FIG. 1 is a diagram of some non-limiting embodiments or aspects of an environment 100 in which systems, devices, products, apparatus, and/or methods, as described herein, may be implemented is shown. As shown in FIG. 1, environment 100 may include flow sensor system 150 including flow sensor 160 and base 180, medical device 102 (e.g., a syringe, etc.) including short range wireless communication tag 104, flexible tubing or IV line 106, communications network 108, and/or remote computing device 110. For example, flow sensor system 150 may include two main assemblies which fit together prior to use: flow sensor 160 and base 180. In some non-limiting embodiments or aspects, flow sensor system 150 includes a flow sensor system as described in U.S. Provisional Ser. No. 63/589,132 , filed Oct. 10, 2023, or U.S. Provisional Ser. No. 63/589,115 , filed on Oct. 10, 2023, which are incorporated herein by reference in their entirety.

Medical device 102 (e.g., a syringe, etc.) may be configured to physically connect and/or fluidically couple to flow sensor 160 as described in more detail herein. Short range wireless communication tag 104 may be attached to or integrated with medical device 102 as described in more detail herein. In some non-limiting embodiments or aspects, short range wireless communication tag 104 includes one or more computing devices, chips, contactless transmitters, contactless transceivers, NFC transmitters/receivers, RFID transmitters/receivers, contact based transmitters/receivers, and/or the like. In some non-limiting embodiments or aspects, short range wireless communication tag 104 can include one or more devices capable of transmitting and/or receiving information to and/or from base 180 via short range wireless communication connection (e.g., a communication connection that uses NFC protocol, a communication connection that uses Radio-frequency identification (RFID), a communication connection that uses a Bluetooth® wireless technology standard, and/or the like).

Flow sensor 160 may be configured to be removably, detachably, physically, and/or electrically connected to base 180 as described in more detail herein. In some non-limiting embodiments or aspects, flow sensor 160 may be connected in-line with an IV line between a fluid source and a patient. In some non-limiting embodiments or aspects, flow sensor 160 may be a single-use flow sensor which is engageable with reusable base 180. Further details regarding non-limiting embodiments or aspects of flow sensor 160 are provided herein below.

Base 180 may be configured to be removably, physically, and/or electrically connected to flow sensor 160 as described in more detail herein. Base 180 may include one or more devices capable of receiving information and/or data from remote computing device 110 (e.g., via communication network 108, etc.) and/or communicating information and/or data to remote computing device 110 (e.g., via communication network 108, etc.). For example, base 180 may include a computing device, a mobile device, and/or the like. In some non-limiting embodiments or aspects, base 180 includes one or more computing devices, chips, contactless transmitters, contactless transceivers, NFC transmitters/receivers, RFID transmitters/receivers, contact based transmitters/receivers, and/or the like. In some non-limiting embodiments or aspects, base 180 can include one or more devices capable of transmitting and/or receiving information to and/or from short range wireless communication tag 104 via a short range wireless communication connection (e.g., a communication connection that uses NFC protocol, a communication connection that uses Radio-frequency identification (RFID), a communication connection that uses a Bluetooth® wireless technology standard, and/or the like). In some non-limiting embodiments or aspects, base 180 includes an integrated power source (not shown), such as a battery, and/or the like. Further details regarding non-limiting embodiments or aspects of base 180 are provided herein below.

Communication network 108 may include one or more wired and/or wireless networks. For example, communication network 108 may include a cellular network (e.g., a long-term evolution (LTE) network, a third generation (3G) network, a fourth generation (4G) network, a fifth generation network (5G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, and/or the like, and/or a combination of these or other types of networks.

Remote computing device 110 may include one or more devices capable of receiving information and/or data from base 180 (e.g., via communication network 108, etc.) and/or communicating information and/or data to base 180 (e.g., via communication network 108, etc.). For example, remote computing device 110 may include a computing device, a server, a group of servers, a mobile device, a group of mobile devices, and/or the like.

The number and arrangement of devices and systems shown in FIG. 1 is provided as an example. There may be additional devices and/or systems, fewer devices and/or systems, different devices and/or systems, or differently arranged devices and/or systems than those shown in FIG. 1. Furthermore, two or more devices and/or systems shown in FIG. 1 may be implemented within a single device and/or system, or a single device and/or system shown in FIG. 1 may be implemented as multiple, distributed devices and/or systems. Additionally, or alternatively, a set of devices and/or systems (e.g., one or more devices or systems) of environment 100 may perform one or more functions described as being performed by another set of devices and/or systems of environment 100.

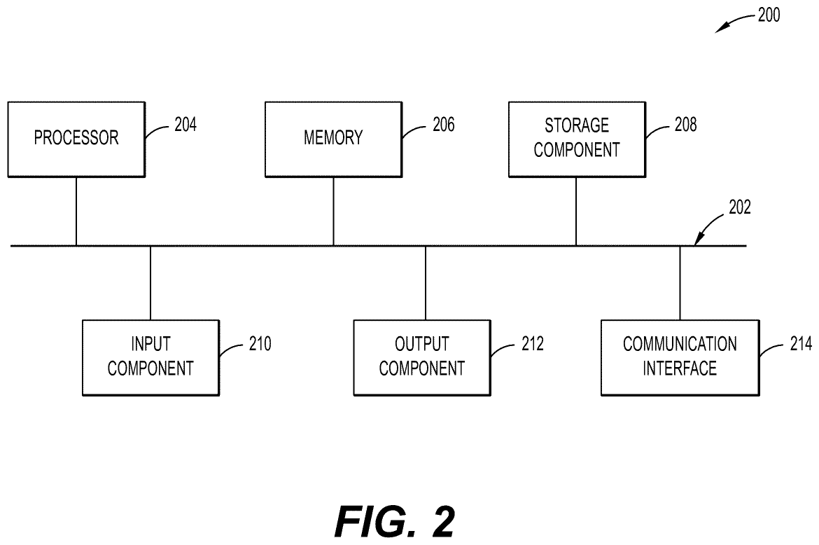

Referring now to FIG. 2, FIG. 2 is a diagram of example components of a device 200. Device 200 may correspond to base 180 and/or remote computing device 110. In some non-limiting embodiments or aspects, base 180 and/or remote computing device 110 may include at least one device 200 and/or at least one component of device 200. As shown in FIG. 2, device 200 may include bus 202, processor 204, memory 206, storage component 208, input component 210, output component 163, and/or communication interface 214.

Bus 202 may include a component that permits communication among the components of device 200. In some non-limiting embodiments or aspects, processor 204 may be implemented in hardware, firmware, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.), and/or the like, which can be programmed to perform a function. Memory 206 may include a random-access memory (RAM), a read only memory (ROM), and/or another type of dynamic or static storage device (e.g., a flash memory, a magnetic memory, an optical memory, etc.) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid-state disk, etc.), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touch screen display, a keyboard, a keypad, a mouse, a button, a switch, a microphone, etc.). Additionally, or alternatively, input component 210 may include a sensor for sensing information (e.g., a global positioning system (GPS) component, an accelerometer, a gyroscope, an actuator, an NFC sensor, an RFID sensor, an optical sensor, a barcode reader, etc.). Output component 163 may include a component that provides output information from device 200 (e.g., a display, a speaker, one or more light-emitting diodes (LEDs), etc.).

Communication interface 214 may include a transceiver-like component (e.g., a transceiver, a separate receiver and transmission source, etc.) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi interface, a cellular network interface, and/or the like.

Device 200 may perform one or more processes described herein. Device 200 may perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally, or alternatively, hardwired circuitry may be used in place of or in combination with software instructions to perform one or more processes described herein. Thus, some non-limiting embodiments or aspects described herein are not limited to any specific combination of hardware circuitry and software.

Memory 206 and/or storage component 208 may include data storage or one or more data structures (e.g., a database, etc.). Device 200 may be capable of receiving information from, storing information in, communicating information to, or searching information stored in the data storage or one or more data structures in memory 206 and/or storage component 208.

The number and arrangement of components shown in FIG. 2 are provided as an example. In some non-limiting embodiments or aspects, device 200 may include additional components, fewer components, different components, or differently arranged components than those shown in FIG. 2. Additionally, or alternatively, a set of components (e.g., one or more components) of device 200 may perform one or more functions described as being performed by another set of components of device 200.

Flow sensor system 150 may reduce medication error at bedside during bolus delivery. Flow sensor system 150 may provide a record of and electronically measure bolus delivery, which allows monitoring bolus delivery and automatic documentation of bolus delivery as part of a patient's health record. Flow sensor system 150 may provide alerts when bolus delivery inconsistent with a patient's medical record is about to occur.

Flow sensor system 150 may be a handheld instrument injection site with interactive interface for syringe injection IV drug delivery and direct electronic medical record documentation. Base 180 may include a durable reusable reader base with a touchscreen display and a separate disposable consumable flow sensor 160.

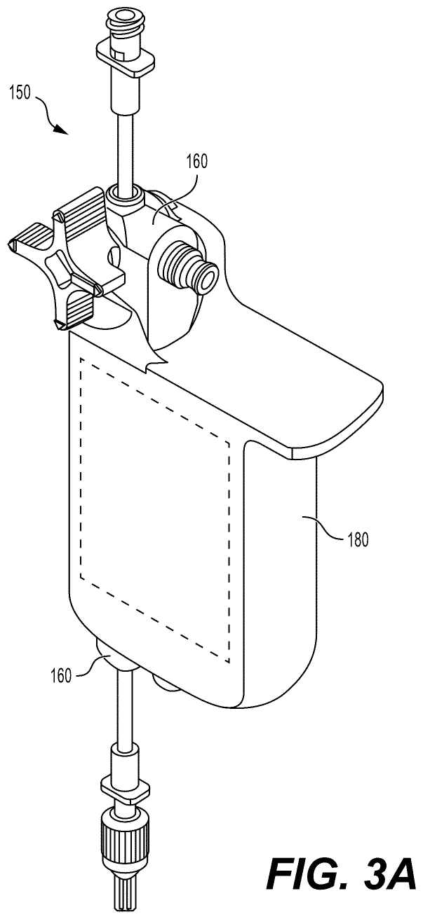

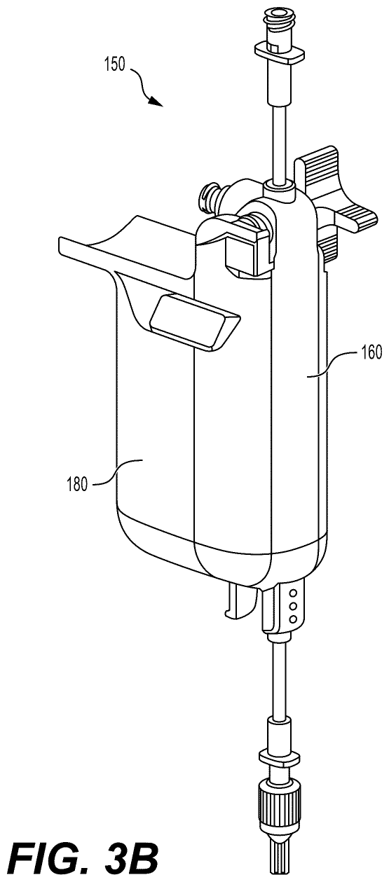

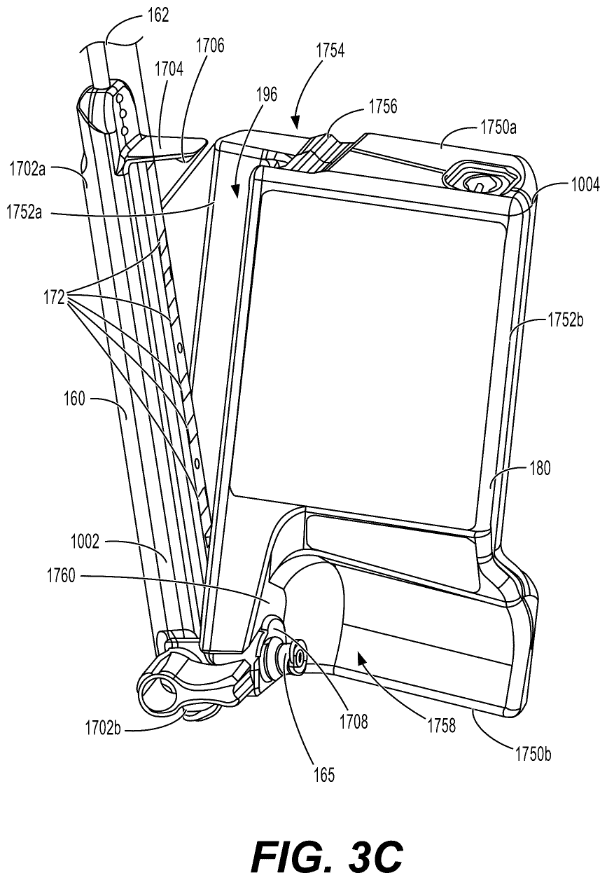

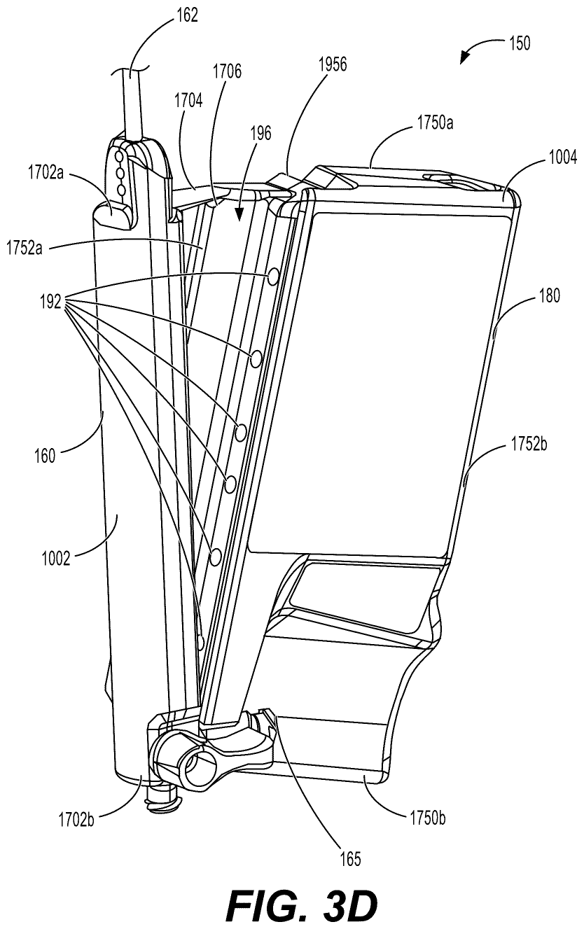

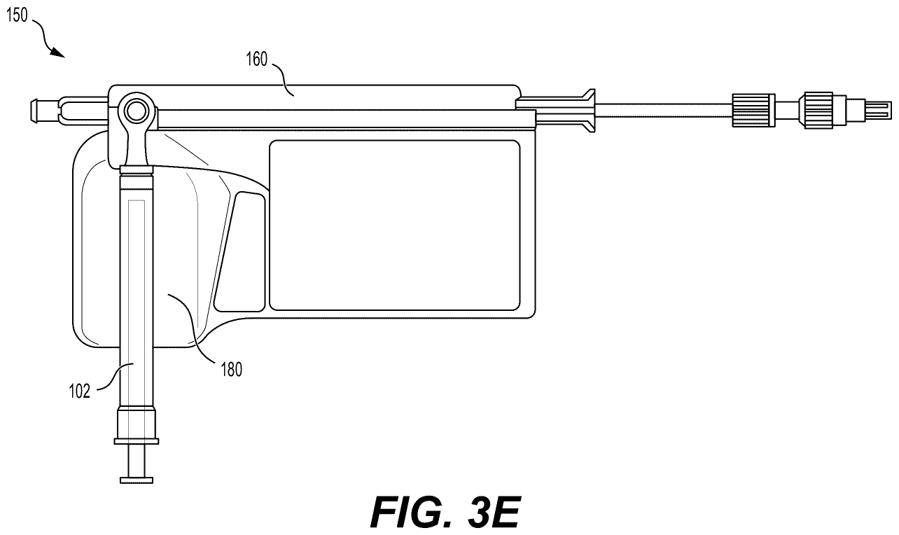

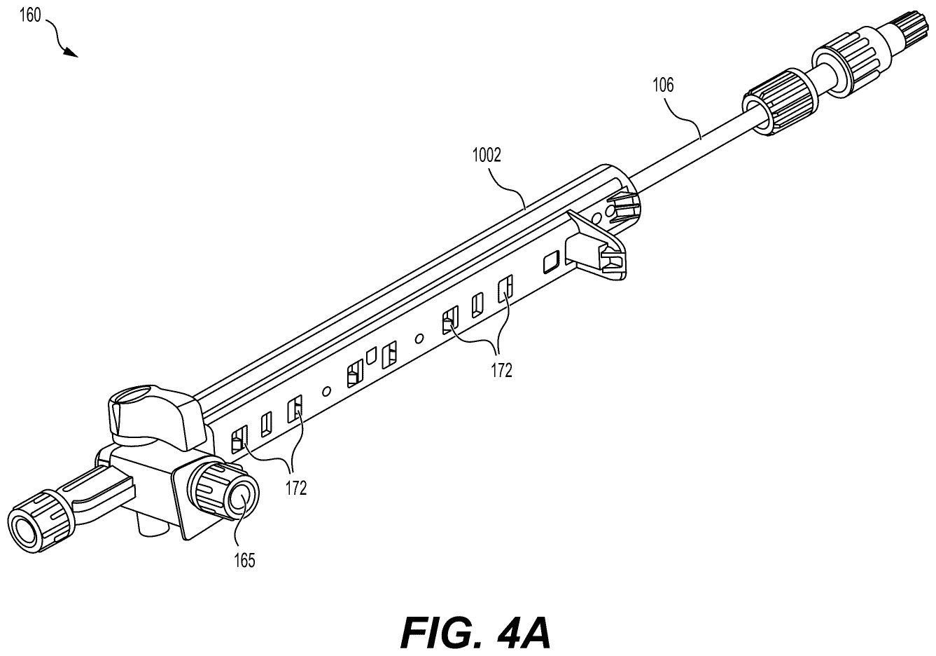

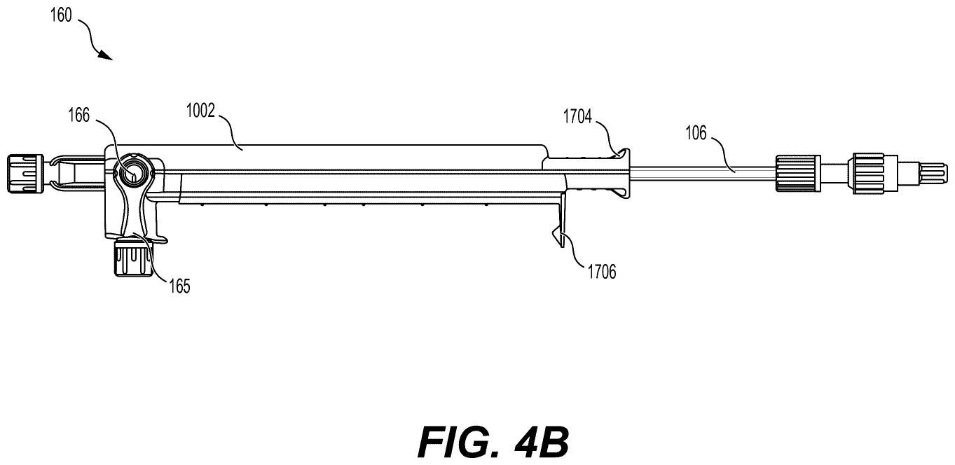

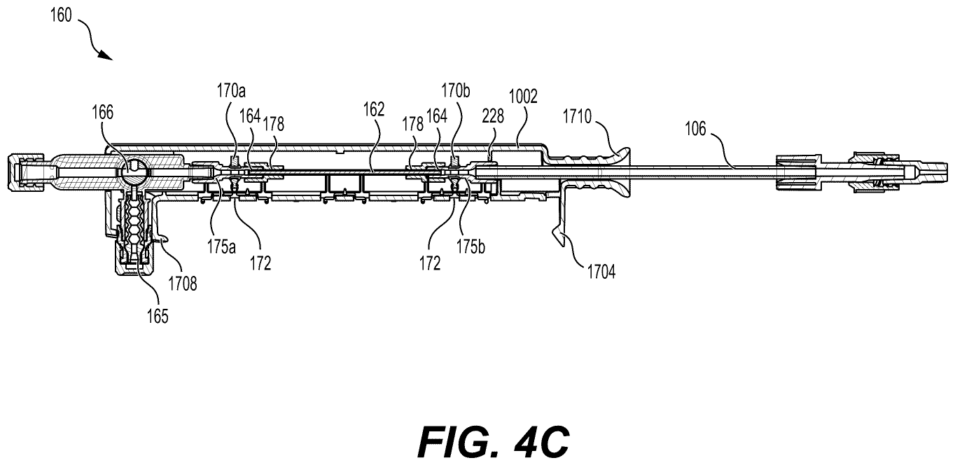

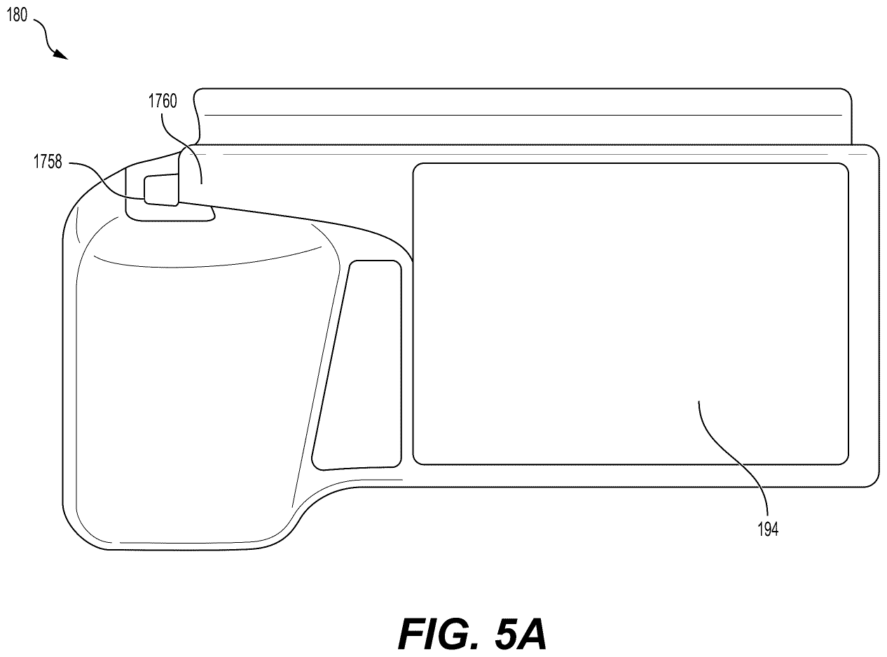

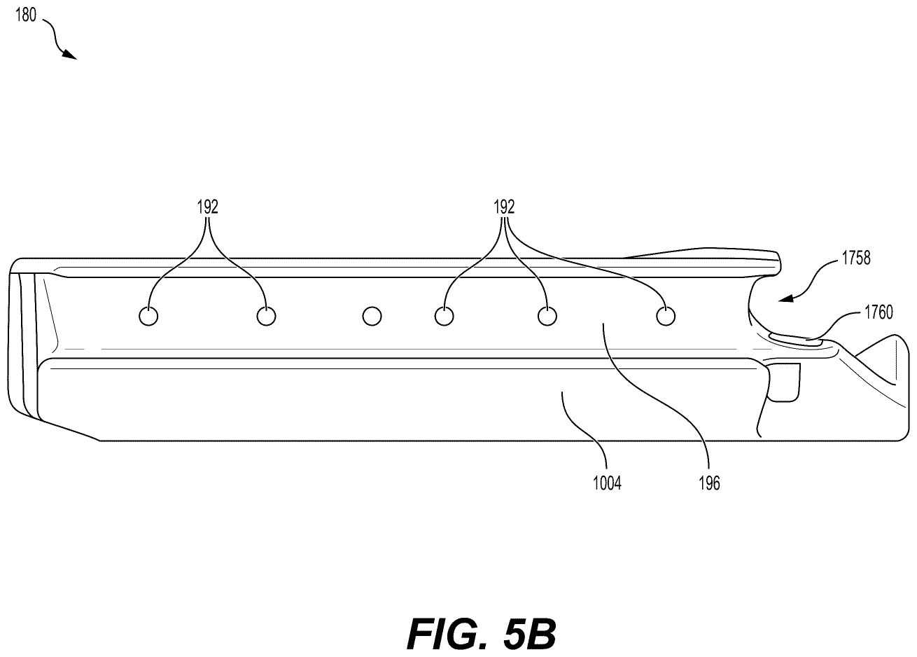

Referring now to FIGS. 3A-3E, 4A-4C, 5A, and 5B, flow sensor 160 may include flow sensor housing 1002 that extends between a first end and a second end opposite the first end. Flow sensor 160 may include a fluid flow path that may be defined at least in part by flow tube 162. Flow tube 162 may include fluid inlet 163 at a first end of flow tube 162 and fluid outlet 164 at a second end of flow tube 162 opposite the first end of flow tube 162. Flow sensor 160 may include fluid injection port 165 and valve 166 (e.g., a manual stopcock valve, etc.) configured to control a flow of a fluid in the fluid flow path of flow sensor 160.

Flow sensor 160 may include at least one sensor 170a, 170b configured to characterize at least one attribute of a fluid in the fluid flow path of flow sensor 160 (e.g., in flow tube 162, etc.). Flow sensor electrical contact 172 (e.g., a plurality of flow sensor electrical contacts 172, etc.) may be in electrical communication with the at least one sensor 170a, 170b. The at least one sensor 170a, 170b may include first ultrasonic transducer or piezoelectric element 170a arranged at an upstream position of the fluid flow path of flow sensor 160 and second ultrasonic transducer or piezoelectric element 170b arranged at a downstream position of the fluid flow path of flow sensor 160. First and second ultrasonic transducers or piezoelectric elements 170a, 170b may be configured to transmit a flow signal indicative of a flow of a fluid (e.g., a flow rate of a fluidic medicament, etc.) in the fluid flow path of flow sensor 160 (e.g., in flow tube 162, etc.). First ultrasonic transducer or piezoelectric element 170a and second ultrasonic transducer or piezoelectric element 170b may be annular in shape and encircle the fluid flow path of flow sensor 160 at respective mounting points a pre-selected distance from each other.

Flow sensor 160 may include inlet fitting or coupler 175a secured to fluid inlet 163 of flow tube 162 and outlet fitting or coupler 175b secured to fluid outlet 214 of flow tube 162. For example, inlet fitting or coupler 175a may be secured at a first end to fluid inlet 163 of flow tube 162 and at a second end opposite the first end to an outlet of valve 166, and/or outlet fitting or coupler 175b may be secured at a first end to fluid outlet 164 of flow tube 162 and at a second end to flexible tubing 106. For example, flow tube 162 may be in fluid communication with valve 166 via inlet fitting 175a, and/or flow tube 162 may be in fluid communication with flexible tubing 106 via outlet fitting or coupler 175b. As an example, flexible tubing 106 may be fixedly coupled to the second end of outlet fitting or coupler 175b. Valve 166 may be configured transition between a plurality of different states to control at least one of: the flow of the fluid between the fluid inlet 163 and the fluid outlet 164, the flow of the fluid between the fluid inlet 163 and the fluid injection port 165, the flow of the fluid between the fluid injection port 165 and the fluid outlet 164, or any combination thereof. For example, valve 166 may include a 3-way stopcock valve, and/or the like. Fluid injection port 165 of flow sensor 160 may extend from flow tube 162 in a first direction parallel to a longitudinal axis of the fluid injection port 165.

First ultrasonic transducer or piezoelectric element 170a may be secured to or mounted on inlet fitting or coupler 175a and/or second ultrasonic transducer or piezoelectric element 170b may be secured to or mounted on outlet fitting or coupler 175b. For example, first ultrasonic transducer or piezoelectric element 170a may be annular in shape and encircle inlet fitting or coupler 175a and/or second ultrasonic transducer or piezoelectric element 170b may be annular in shape and encircle outlet fitting or coupler 175b. At least one absorber sleeve 178 may be engaged with (e.g., directly engaged with, etc.) and/or encircle inlet fitting or coupler 175a, flow tube 162, and/or outlet fitting or coupler 175b.

First and second ultrasonic transducers or piezoelectric elements 170a and 170b may be in electrical communication with one or more processors of base 180 when flow sensor 160 is connected to base 180. For example, base 180 may interact with first and second ultrasonic transducers or piezoelectric elements 170a and 170b in flow sensor 160 to measure displacement of fluid through flow sensor 160. For example, flow sensor electrical contact 172 may be in electrical communication with a corresponding base electrical contact when flow sensor 160 is connected (e.g., connected, attached, mounted, etc.) to base 180.

Base 180 may include one or more processors 204, base electrical contact 192 (e.g., a plurality of base electrical contacts 192, a plurality of base electrical contacts 192 corresponding to the plurality of flow sensor electrical contacts, etc.) in electrical communication with the one or more processors 204, a short range wireless communication device (e.g., communication interface 214, a near-field communication (NFC) receiver, etc.), and/or a display 194 (e.g., input component 210, output component 212, a touchscreen display configured to receive user input from a user, etc.). The flow sensor electrical contact 172 may be in electrical communication with the base electrical contact 192 when the flow sensor 160 is connected (e.g., connected, attached, mounted, etc.) to the base 180. For example, the plurality of flow sensor electrical contacts 172 may be in electrical communication with corresponding ones of the plurality of base electrical contacts 192 when the flow sensor 160 is connected (e.g., connected, attached, mounted, etc.) to the base 180. As an example, the flow sensor electrical contact 172 may include a plurality of electrical contacts spaced apart from each other along flow sensor housing 1002. As an example, base electrical contact 192 may include a plurality of electrical contacts spaced apart from each other along base housing 1004 within opening 196 between the first end 1750a and the second end 1750b of base housing 1004.

Base 180 may include slot or opening 196 configured to receive the flow sensor 160, and flow sensor 160 may be configured for sliding engagement and/or snap-fit connection with slot or opening 196 of the base 180. For example, slot or opening 196 of base 180 may include base electrical contact 192 (e.g., a plurality of base electrical contacts 192, a plurality of base electrical contacts 192 corresponding to the plurality of flow sensor electrical contacts 172, etc.).

Display 194 may include a touchscreen display configured to receive user input from a user. For example, display 194 may include an interactive graphical user interface configured to display a current status of internal functions of base 180, a current status of an injection site, and/or a prompt for user interaction, and base 180 may interact with the user via touchscreen display, audio, voice command, haptic feedback, and/or the like (e.g., to prompt the user on current status and request user input, etc.). Accordingly, by incorporating display 194 into base 180, a user need not remove their attention from the base 180 to interact with the display 194.

Base 180 may include a wireless communication device configured to communicate information associated with the at least one attribute of the fluid in the flow tube 162 to remote computing device 110. For example, base 180 may communicate information and/or data with remote computing device 110 to document drug delivery occurrences into patient medical records (e.g., patient medical records associated with a patient wristband bar code label scanned by the optical scanner of base 180, etc.).

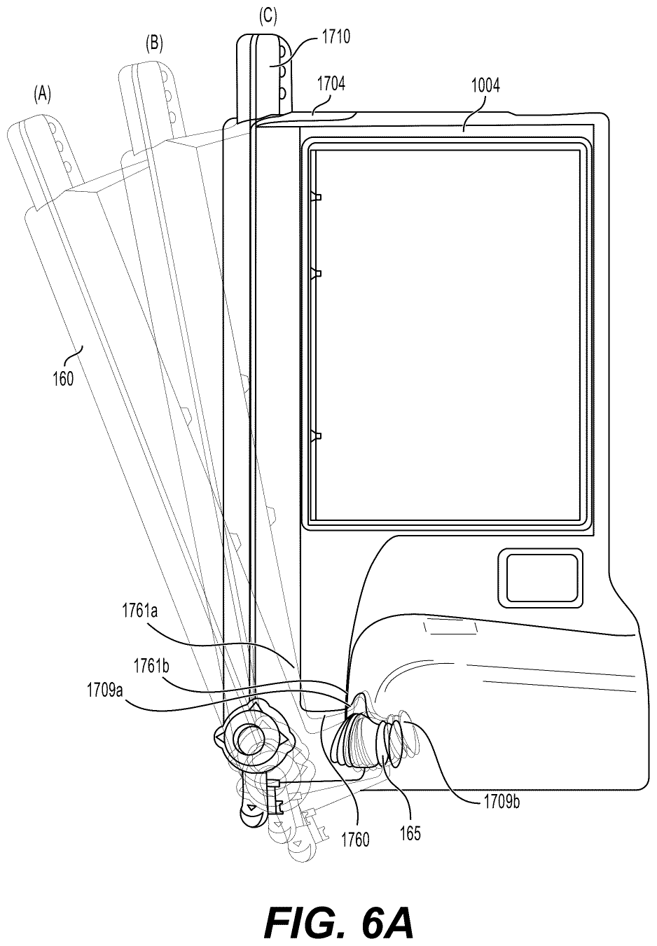

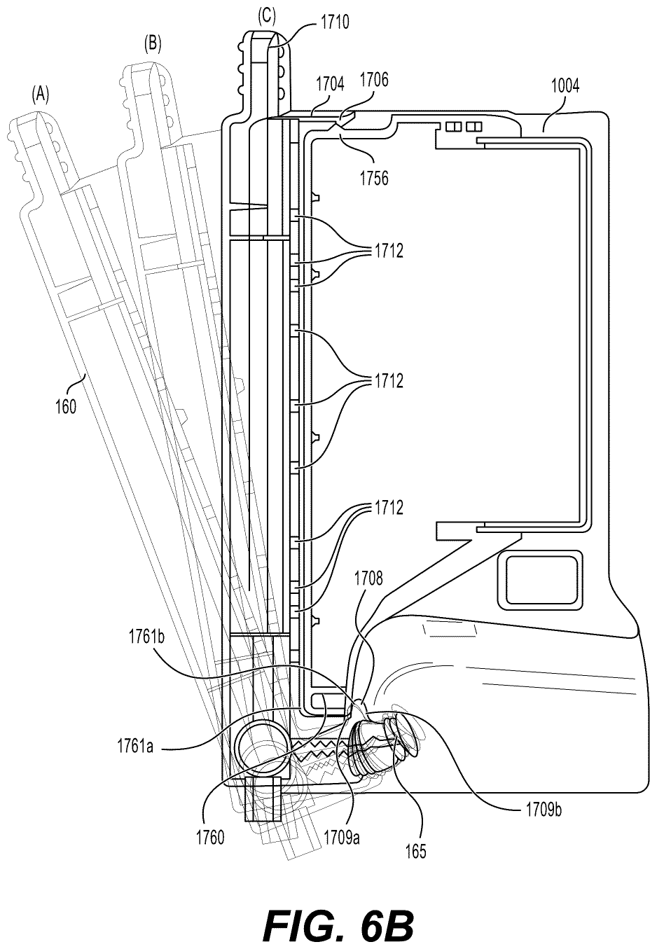

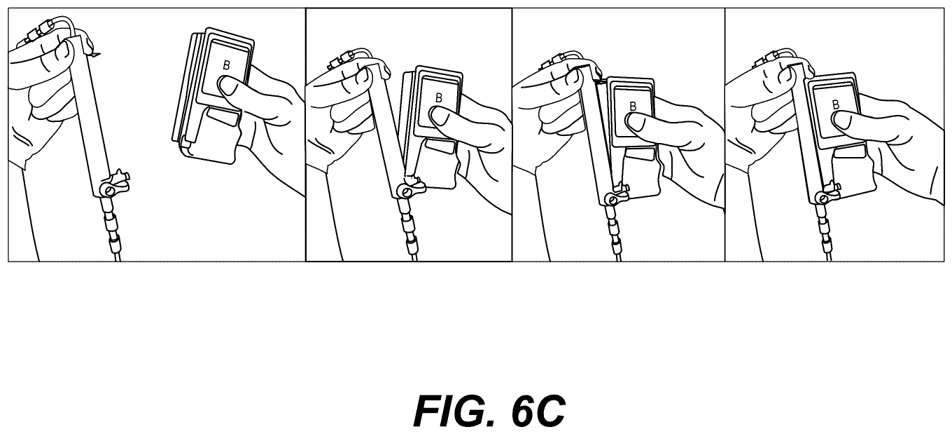

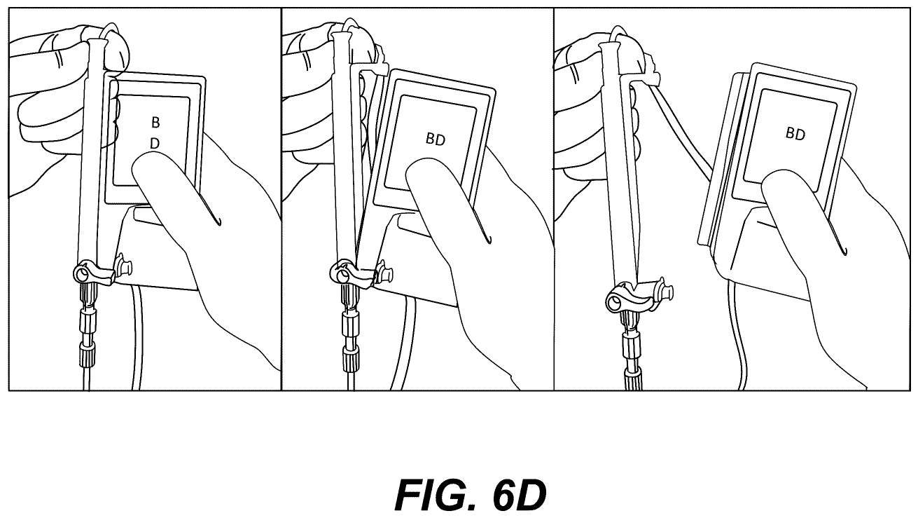

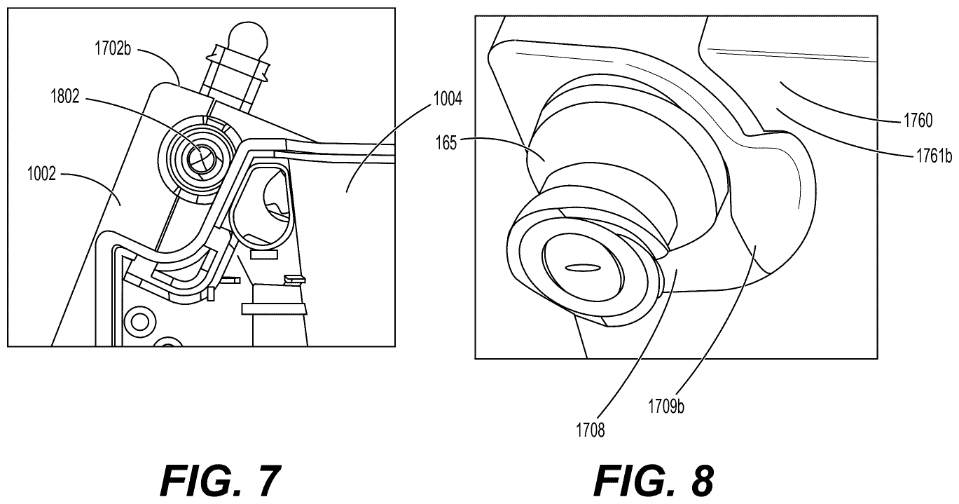

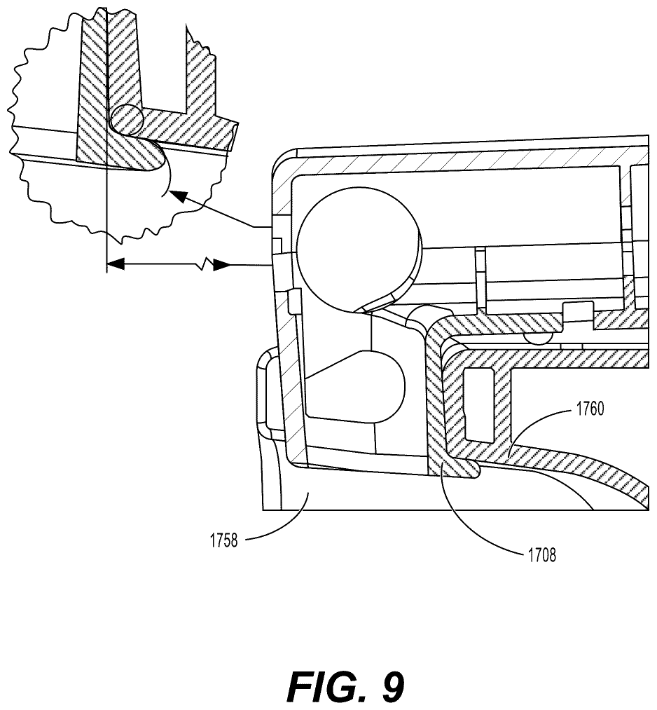

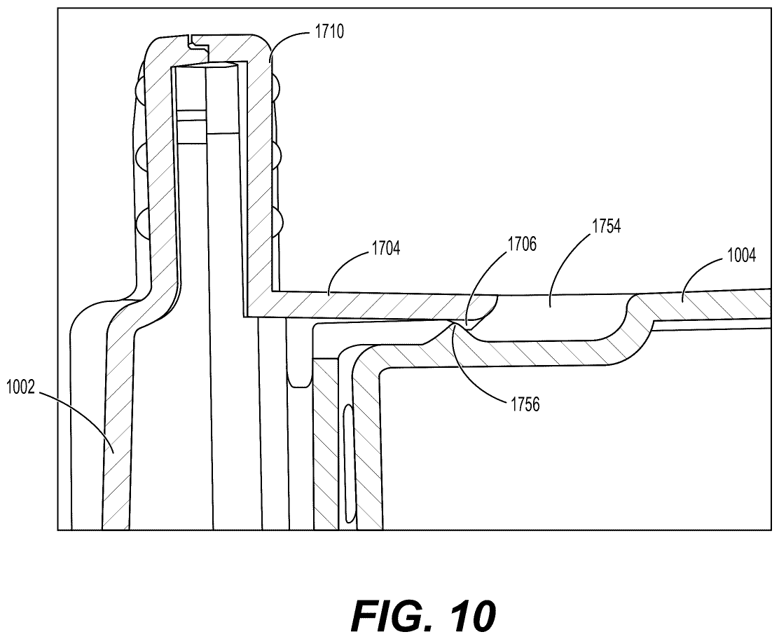

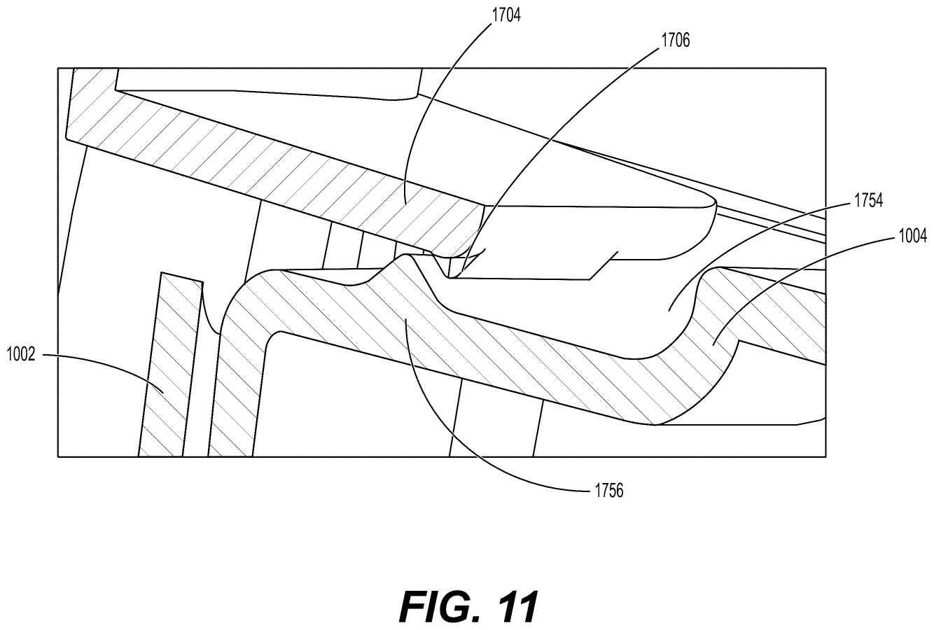

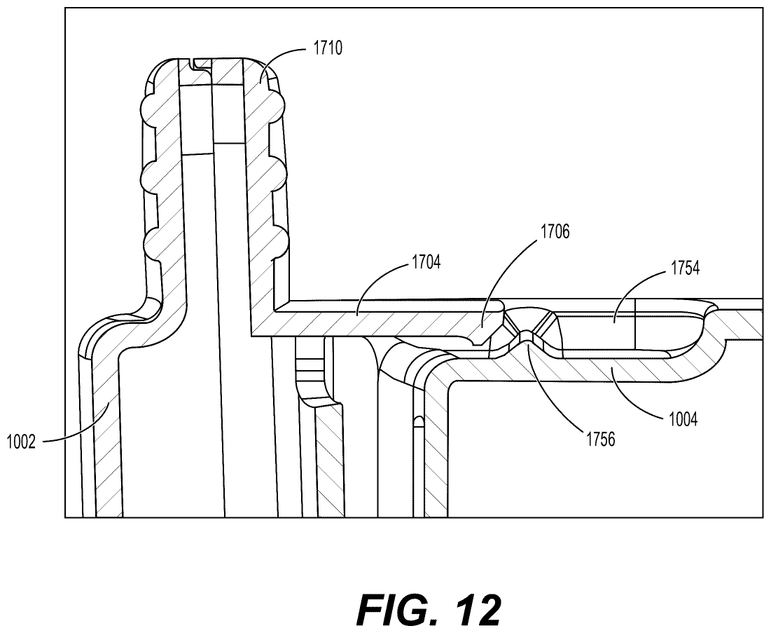

Still referring to FIGS. 3A-3E, 4A-4C, 5A and 5B, and referring also to FIGS. 6A-6D and 7-12, in some non-limiting embodiments or aspects, flow sensor 160 and base 180 may be detachably connectable to each other (e.g., via a snap-fit connection, etc.) between flow sensor housing 1002 and base housing 1004. For example, flow sensor housing 1002 may extend between a first end 1702a and a second end 1702b opposite the first end 1702a. Flow sensor housing 1002 may include beam 1704 that extends from flow sensor housing 1002 at the first end 1702a of flow sensor housing 1002. Beam 1704 may include beam tab 1706 that extends from beam 1704. Beam 1704 and beam tab 1706 may be configured to form the snap-fit connection with base housing 1004 as described in more detail herein below and in in U.S. Provisional Patent Application No. 63/589,132 , filed Oct. 10, 2023, or U.S. Provisional Patent Application 63/589,115, filed on Oct. 10, 2023, which are incorporated herein by reference in their entirety.

In some non-limiting embodiments or aspects, flow sensor housing 1002 and/or components thereof may include or be composed of a polycarbonate material, such as Makrolon® Rx2530 (Gray 90, PC) manufactured by Covestro AG. In some non-limiting embodiments or aspects, base housing 1004 and/or components thereof may include or be composed of a polycarbonate material, such as Makrolon Rx3440 (Gray 70, PC) manufactured by Covestro AG.

Fluid injection port 165 may extend from the flow sensor housing 1002 at the second end 1702b of the flow sensor housing 1002 in a same direction as beam 1704. For example, flow sensor housing 1002 may extend from the second end 1702b toward the first end 1702a in a first direction, beam 1704 may extend from flow sensor housing 1002 in a second direction at least partially perpendicular to the first direction, and/or fluid injection port 165 may extend from flow sensor housing 1002 in the second direction at least partially perpendicular to the first direction.

Flow sensor housing 1002 may include first arced flange 1708 that at least partially surrounds fluid injection port 165.

The first end 1702a of flow sensor housing 1002 may include protruding grip 1710 extending from the first end 1702 of the flow sensor housing 1002 away from beam 1704. For example, protruding grip 1710 may extend from the first end 1702 of the flow sensor housing 1002 away from beam 1704 in the first direction. As an example, flow tube 162 may pass through an opening in protruding grip 1710 and extend through an interior of protruding grip 1710 and flow sensor housing toward the second end 1702b of flow sensor housing 1002 where it may exit flow sensor housing 1002 via another opening in flow sensor housing 1002.

Flow sensor housing may include a plurality of bosses 1712 extending from flow sensor housing 1002 between beam 1704 and fluid injection port 165. For example, the plurality of bosses 1712 may be spaced apart from one another along the outer surface of flow sensor housing 1002. As an example, the plurality of bosses 1712 may extend from flow sensor housing 1002 in a same direction as beam 1704 and/or fluid injection port 162 (e.g., in the second direction, etc.).

Base housing 1004 may extend between a first end 1750a and a second end 1750b opposite the first end 1750a and between a first side 1752a and a second side 1752b opposite the first side 1752a. The first side 1752a of base housing 1004 may include slot or opening 196 that extends between the first end 1750a and the second end 1750b opposite the first end 1750a. Opening 196 may be configured to receive flow sensor housing 1002. Opening 196 may extend from the first side 1752a at the first end 1750a of base housing 1004 toward the second side 1752b of base housing 1004 as cavity 1754 in the first end 1750a of base housing 1004. Cavity 1754 may include cavity tab 1756 that extends from the first end 1750a of base housing 1004 away from the second end 1750b of base housing 1004 (e.g., in the first direction, etc.). Beam 1704 and beam tab 1706 of flow sensor housing 1002 may be configured to form the snap-fit connection with cavity 1754 and cavity tab 1756 of base housing 1004. For moldability, cavity tab 1756 may be formed by cavity 1754 without a need for a slide, and/or beam tab 1706 may be formed by beam 1704.

Opening 196 may extend from the first side 1752a at the second end 1750b of base housing 1004 toward the second side 1752b of base housing 1004 as an at least partially open bore 1758. The at least partially open bore 1758 may be partially surrounded by second arced flange 1760. Second arced flange 1760 may be configured to receive and partially surround fluid injection port 165 with first arced flange 1708 of flow sensor housing 1002 between second arced flange 1760 of base housing 1004 and the second side 1752b of base housing 1004.

First arced flange 1708 of flow sensor housing 1002 may include a first face 1709a that faces flow sensor housing 1002 and a second face 1709b opposite the first face 1709a (e.g., that faces away from flow sensor housing in the second direction, etc.). Second arced flange 1760 of base housing 1004 may include a first face 1761a that faces the first side 1752a of base housing 1004 and away from the second side 1752b of base housing 1004 and a second face 1761b opposite the first face 1761a that faces toward the second side 1752b of base housing 1004. When fluid injection port 165 is received within and partially surrounded by second arced flange 1760 of base housing 1004, first arced flange 1708 of flow sensor housing 1002 may be rotatable with respect to second arced flange 1760 of base housing 1004 to bring the first face 1709a of first arced flange 1708 of flow sensor housing 1002 into contact with the second face 1761b of second arced flange 1760 of base housing 1004. Accordingly, first arced flange 1708 and second arced flange 1760 may form a tapered dovetail-style joint with an additional retaining tab that reduces a tolerance of the tapered dovetail-style joint and acts as a pivot point and provides positive engagement therebetween. The second arced flange may be configured with generous lead-in angles/drafts for ease of connection.

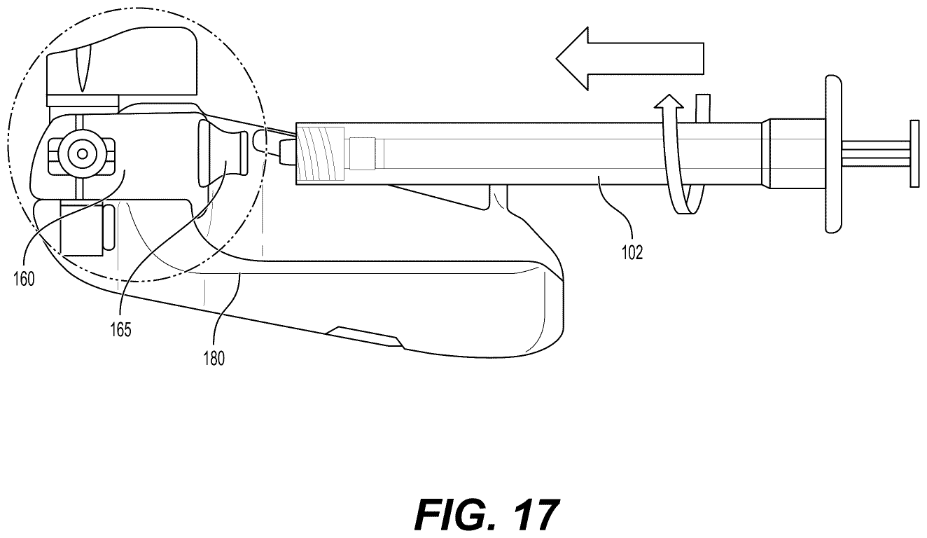

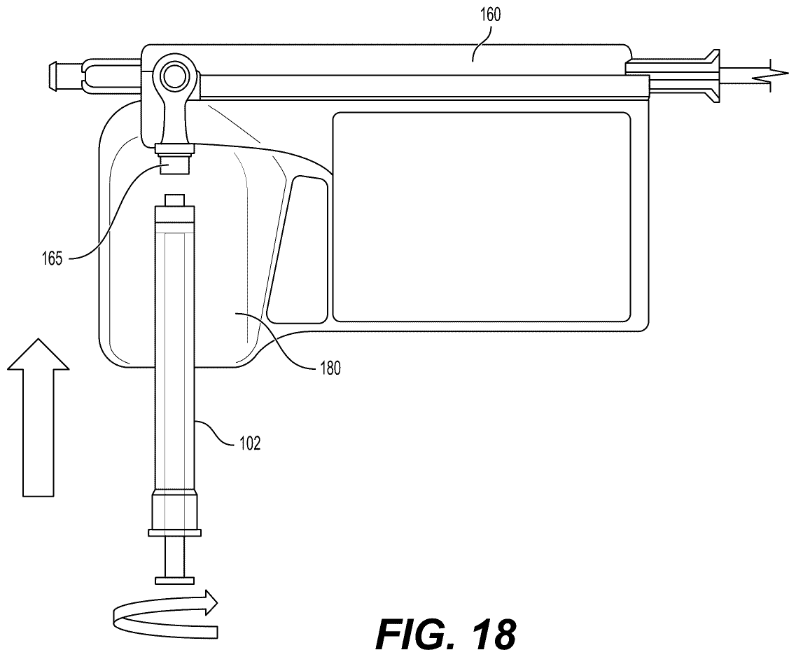

As shown in FIGS. 6A-6C, to connect or attach flow sensor 160 to base 180, fluid injection port 165 may be inserted into second arced flange 1760 of base housing 1004 with first arced flange 1708 of flow sensor housing 1002 located between the second arced flange 1760 and the second side 1750 of base housing 1004 in bore 1758. For example, as shown in Position (A) of FIGS. 17A and 17B, flow sensor housing 1002 of flow sensor 160 may be angled with respect to base housing 1004 of base 180 when fluid injection port 165 is initially inserted into second arced flange 1760 of base housing 1004 with first arced flange 1708 of flow sensor housing 1002 located between the second arced flange 1760 and the second side 1750 of base housing 1004 in bore 1758. As an example, and as shown in FIG. 18, the angle of insertion or rotation of flow sensor housing 1002 of flow sensor 160 with respect to base housing 1004 of base 180 may be defined and/or limited by an interaction between valve position detection component 1802 that extends from flow sensor housing 1002 in a third direction perpendicular to fluid injection port 165 (e.g., perpendicular to the first direction and/or the second direction, etc.) base housing 1004. In such an example, valve position detection component 1802 may include a medical stopcock assembly with position detection as described in U.S. patent application Ser. No. 18/013,392, filed on Jun. 29, 2021, the content of which is hereby incorporated by reference in its entirety.

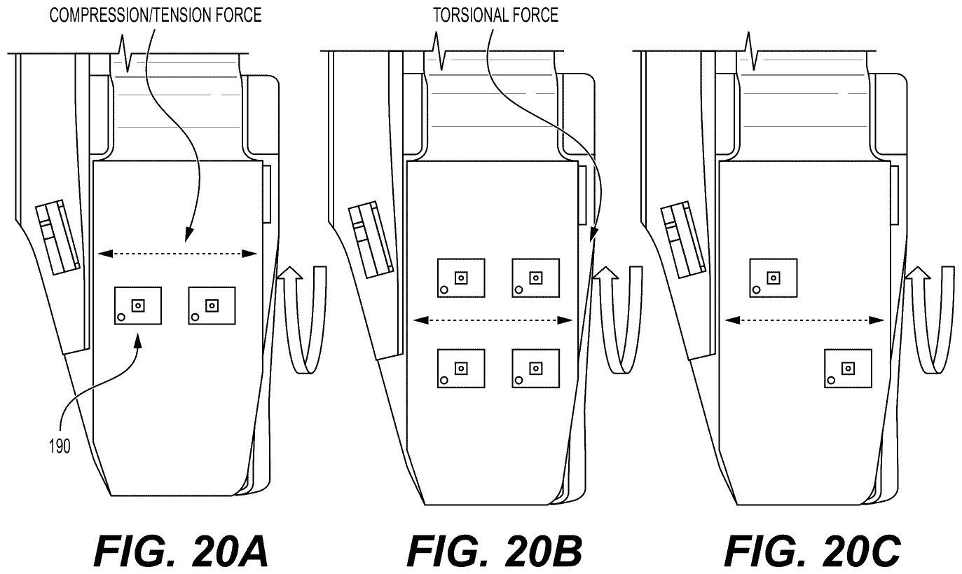

As further shown in FIGS. 6A-6C, flow sensor housing 1002 of flow sensor 160 may be rotated or pivoted with respect base housing 1004 of base 180 from Position (A) through Position (B) to Position (C) in a fist pivot direction to connect or attach flow sensor 160 to base 180. As flow sensor housing 1002 of flow sensor 160 is rotated from Position (B) to Position (C), first arced flange 1708 may be rotated with respect to second arced flange 1760, thereby forming two sliding tangent pivot points as shown in FIG. 20 to retain the second end 1702b of flow sensor housing 1002 in connection with flow sensor base 1004, and/or beam 1704 may be received within cavity 1754 with beam tab 1706 encountering, deflecting, and passing over cavity tab 1756 to form the snap fit connection to secure the first end 1702a of flow sensor housing 1002 to base 1004. The interaction of beam tab 1706 when encountering, deflecting, and passing over cavity tab 1756 may provide an audible snap to indicate a secure connection between flow sensor 160 and base 180. In such an example, beam 1704, beam tab 1706, and cavity tab 1756 may be configured such that less than or equal to 20 N of force applied to protruding grip 1710 in the second direction causes beam tab 1706 to deflect from and pass over cavity tab 1756 to form the snap-fit connection, and which brings flow sensor electrical contact 172 into electrical communication with base electrical contact 192. In such an example, the plurality of bosses 1712 may contact the outer surface of base housing 1004 within opening 196 to reduce or prevent over-travel of flow sensor electrical contact 172 with respect to base electrical contact 192 and/or flow sensor housing 1002 and/or to reduce or minimize a stack-up tolerance as compared to an entire length of flow sensor housing 1002 contacting base housing 1004. A boss 1712 may not be provided nearest the snap-fit connection (e.g., with a threshold distance of beam 1704, etc.) to enable over-travel of beam 1704 for a snap-fit connection with reduced or minimal bending of flow sensor housing 1002 from a fulcrum of a closest middle boss of the plurality of bosses.

Accordingly, and referring specifically to FIG. 6C, flow sensor 160 may be guided into connection with base 180 via the tapered “dovetail” shape of first arced flange 1708 and second arced flange 1756, after which a length of flow sensor housing 1002 may be keyed and guided into opening 196 of base housing 1004 while being supported at the second end 1702b of flow sensor housing 1002. The snap-fit retention of beam 1704 and beam tab 1706 may be guided in by a gradual lead ramp on base housing 1004 at a top of opening 196 that extends into opening 1754. Each of the snap-fit beam 1704, snap bump 1706 and flow sensor housing 1002 may be guided to ensure consistent alignment of the snap-fit mechanism. The snap-fit may be captured by a return angle of cavity tab 1756 which quickly and securely connects flow sensor housing 1002 to base housing 1004. In this way, a user may receive positive audible and tactile feedback. This feedback, along with the lead and return angles of the snap-fit connection may help to indicate that flow sensor 160 is either attached or detached, without ambiguity of successful attachment or detachment.

As further shown in FIGS. 6A, 6B, and 6D, flow sensor housing 1002 of flow sensor 160 may be rotated or pivoted with respect base housing 1004 of base 180 in a second pivot direction opposite the first pivot direction from Position (C) through Position (B) to Position (A) to disconnect or detach flow sensor 160 from base 180. As flow sensor housing 1002 of flow sensor 160 is rotated from Position (C) to Position (B), beam tab 1706 may encounter, deflect, and pass over cavity tab 1756 to release the snap fit connection to detach the first end 1702a of flow sensor housing 1002 from base 1004. The interaction of beam tab 1706 when encountering, deflecting, and passing over cavity tab 1756 may provide an audible snap to indicate a release of the secure connection between flow sensor 160 and base 180. In such an example, beam 1704, beam tab 1706, and cavity tab 1756 may be configured such that less than or equal to 24 N of force applied to protruding grip 1710 in a direction opposite the second direction causes beam tab 1706 to deflect from and pass over cavity tab 1756 to release the snap-fit connection. As flow sensor housing 1002 of flow sensor 160 is rotated from Position (C) to Position (A), first arced flange 1708 may be rotated with respect to second arced flange 1760 to enable fluid injection port 165 to be removed from second arced flange 1760 of base housing 1004 to disconnect or detach second end 1702b of flow sensor housing 1002 of flow sensor 160 to from bases housing 1004 of base 180. Referring now specifically to FIG. 17D, disconnection of flow sensor 160 from base 180 may be achieved via protruding grip 1710 by grasping protruding grip 1710 and swinging protruding grip 1710 away from base 180.

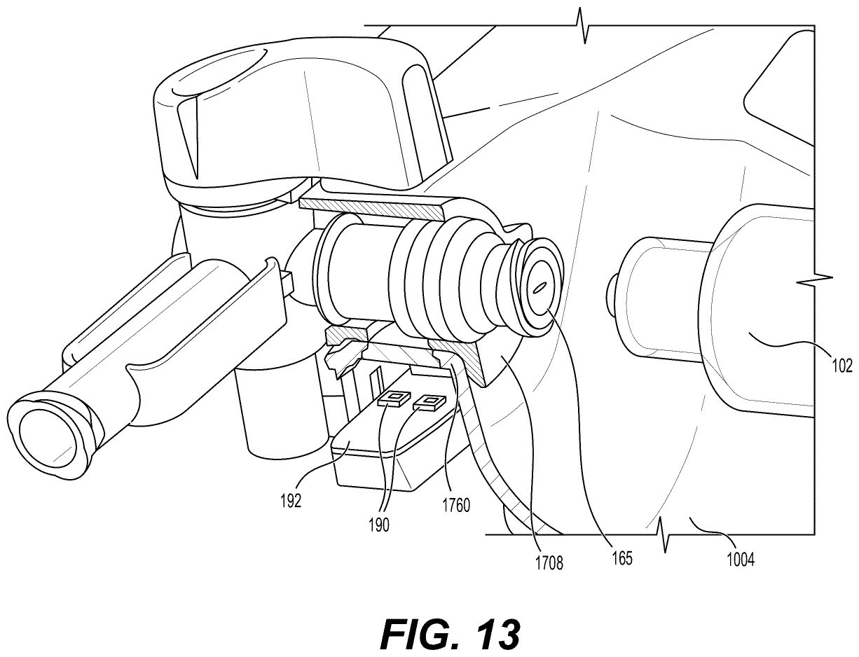

Referring now to FIG. 13, base 180 may include at least one force sensor 190 configured to generate at least one force signal indicative of at least one force applied to the base housing 1004. For example, the at least one force sensor 190 may be housed within base housing 1004. In some implementations, the at least one force sensor 190 may be mounted on PCB 192 in base 180. For example, PCB 192 and/or the at least one force sensor 190 may be located within a portion of base housing 1004 proximate fluid injection port 165 when flow sensor 160 is connected to base 180 (e.g., within a portion of base housing 1004 that forms or defines the at least partially open bore 1758 and/or second arced flange 1760, etc.). As an example, PCB 192 and/or the at least one force sensor 190 may be located within base housing 1004 proximate to second arced flange 1760 of base housing 1004.

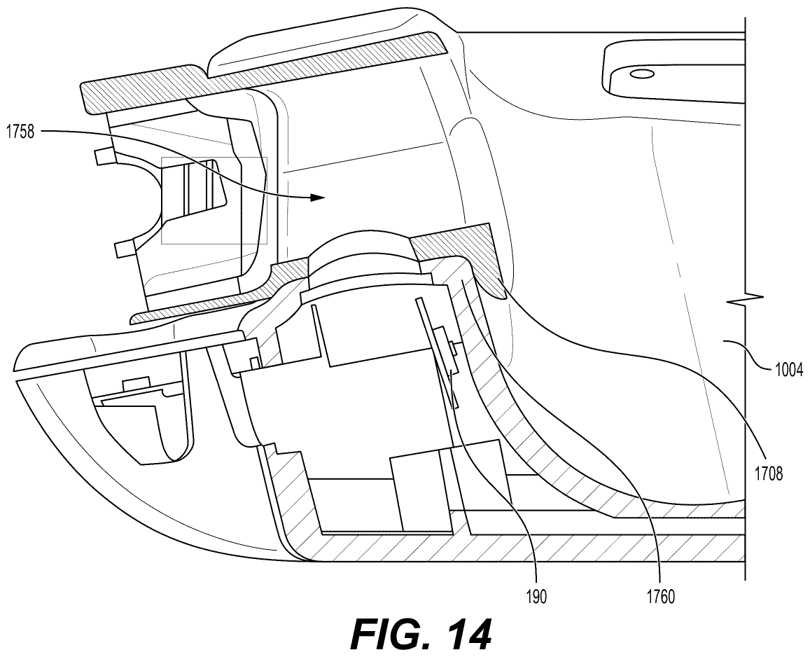

In some implementations, and referring specifically to FIG. 14, the at least one force sensor 190 may be mounted (e.g., on PCB 192, etc.) within base housing 1004 in direct physical contact with an inner wall of base housing 1004. For example, an actuator button of the at least one force sensor 190 may be in direct physical contact with an inner wall of base housing 1004 to directly measure a force applied to base housing 1004. As an example, the at least one force sensor 190 may be mounted (e.g., on PCB 192, etc.) within base housing 1004 directly in-line with a force to be measured (e.g., directly in-line with the compression force resulting from the initial pressing of medical device 102 into fluid injection port may be transferred from flow sensor 160 to base 180, etc.). For example, the at least one force sensor 190 may be in direct physical contact with first face 1761a of second arced flange 1760 of base housing 1004 to receive the force applied to base housing 1004 to generate the at least one force signal indicative of the at least one force applied to base housing 1004.

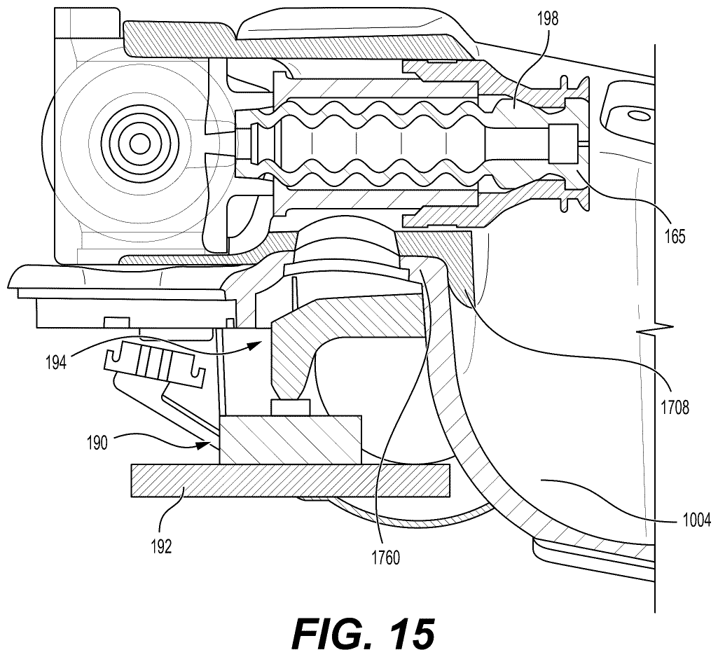

In some implementations, and referring specifically to FIG. 15, base 180 may include at least one actuator arm 194 in physical contact with the at least one force sensor 190 and an inner wall of base housing 1004. The at least one actuator arm 194 may be configured to transfer at least a portion of the force (e.g., the compression force, the torsional force, etc.) applied to base housing 1004 by flow sensor housing 1002 to the at least one force sensor 190. For example, the least one actuator arm 194 may be in physical contact with base housing 1004 at an inner surface of second arced flange 1760. As an example, the at least one force sensor 190 may be configured to receive the force applied to base housing 1004 via second arced flange 1760 of base housing 1004 to generate the at least one force signal indicative of the at least one force applied to base housing 1004. For example, the at least one force sensor 190 may be configured to receive the at least one force applied to base housing 1004 via first face 1761a of second arced flange 1760 of base housing 1004 to generate the at least one force signal indicative of the at least one force applied to base housing 1004.

The one or more processors 204 of base 180 may be configured to determine, based on the at least one force signal, a connection of medical device 102 (e.g., a syringe, etc.) to fluid injection port 165 of flow sensor 160 or a disconnection of medical device 102 (e.g., the syringe, etc.) from fluid injection port 165 of flow sensor 160. In some implementations, the one or more processors 204 of base 180 may be further configured to determine, based on the at least one force signal, a connection of flow sensor 160 to base 180 or a disconnection of flow sensor 160 from base 180. For example, the one or more processors 204 of base 180 may be configured to determine, based on the at least one force signal, a connection of medical device 102 (e.g., a syringe, etc.) to fluid injection port 165 of flow sensor 160, a disconnection of medical device 102 (e.g., the syringe, etc.) from fluid injection port 165 of flow sensor 160, a connection of flow sensor 160 to base 180, and/or a disconnection of flow sensor 160 from base 180.

The at least one force sensor 190 may include a force sensor, a pressure sensor, a strain gauge, a load cell, a force gauge, and/or the like. A force sensor may undergo a change in electrical voltage when subjected to a load. For example, when a force is exerted on a piezoelectric crystal of a force sensor, the force sensor may generate an electrical signal proportional to the applied load, which enables precise force detection and analysis. As an example, a force sensor may include a piezoelectric crystal equipped with an actuator button and electrical contacts. For example, when integrated into or connected with an electrical monitoring circuit (e.g., to the one or more processors 204 of base 180, etc.), the force sensor may be configured to continuously monitor a force applied to the actuator button, which enables precise measurement and monitoring of forces. As an example, a compatible digital controller (e.g., an application-specific integrated circuit (ASIC), etc.) may be configured to digitally connect to and monitor one or more force detectors. By calibrating the applied force, the compatible digital controller may provide and/or use a digital output for each force detector, enabling accurate monitoring of any changes in the applied force, as well as precise and reliable force measurements. In some implementations, a force sensor may be surface mounted on PCB 194 and/or packaged with a center point at which a force is applied. Electrical contacts points of the force sensor may be monitored across the piezoelectric device, with distortion resulting from an applied force to the actuator button changing an output voltage or impedance of the force sensor. In some implementations, the one or more processors 204 of base 180 may be configured to monitor the output voltage or impedance between each electrical contact of a force sensor.

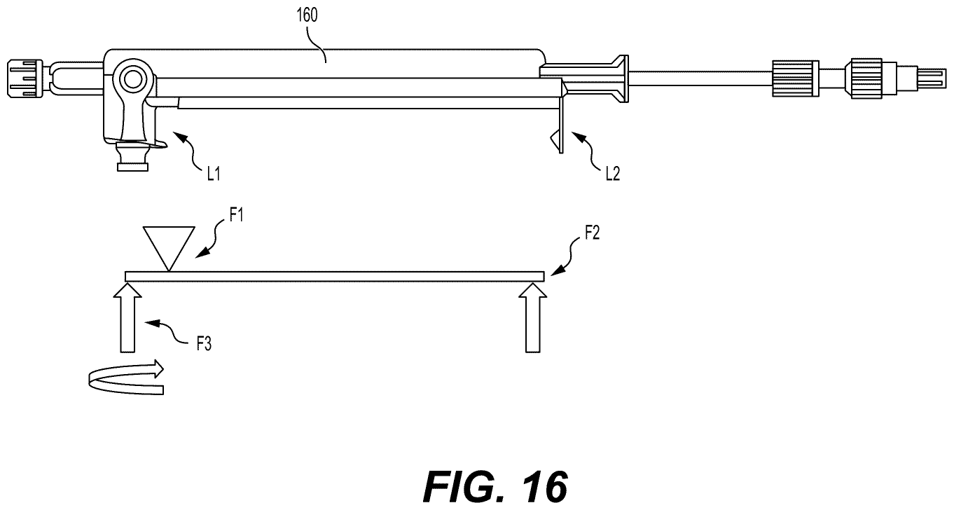

As previously described herein with respect to FIGS. 6A-6D, a process of mounting flow sensor 160 to base 180 may include snapping flow sensor 160 into or onto base 180. For example, and referring now specifically to FIG. 16, flow sensor 160 may be mounted to base 180 by engaging latches L1 and L2 at opposing ends of flow sensor 160 to base 180. This process may exert forces onto base 180 at the two attachment or latching points L1 and L2 including a pivot point at the first side 1752a at the second end 1750b of base housing 1004 of base 180 and a retaining latch/snap at the first side 1752a at the first end 1750a of base housing 1004 of base 180. As flow sensor 160 is attached to base 180, a reaction force F1 may be applied at the interface between first arced flange 1708 of flow sensor housing 1002 and second arced flange 1760 of base housing 1004, resulting in compression between flow sensor housing 1002 and base housing 1004. Beam 1704 and beam tab 1706 may form the snap-fit connection with cavity 1754 and cavity tab 1756 of base housing 1004, which may apply a force F2 of approximately <10 N to attach flow sensor 160 to base 180, and detaching flow sensor 160 from base 180 may use a force between 8 N and 24 N to undo the snap-fit connection. The at least one force sensor 190 may measure these initial forces resulting from the connection of flow sensor 160 to base 180, and the one or more processors 204 of base 180 may process these measured initial forces to establish a baseline measurement as a signature pattern for a connection of flow sensor 160 to base 180, and the baseline force signature may be used to compare actual flow sensor connection or disconnection events to determine flow sensor connection state. In some implementations, analyzing the at least one force signal includes providing the at least one force signal to a machine-learning model trained to identify flow sensor connection or disconnection events. For example, the one or more processors 204 of base 180 and/or remote computing system 110 may analyze the at least one force signal to determine the flow sensor connection state by providing data associated with the at least one force signal as input to the machine learning model trained to determine the flow sensor connections state and receiving as output from the machine learning model the flow sensor connection state.

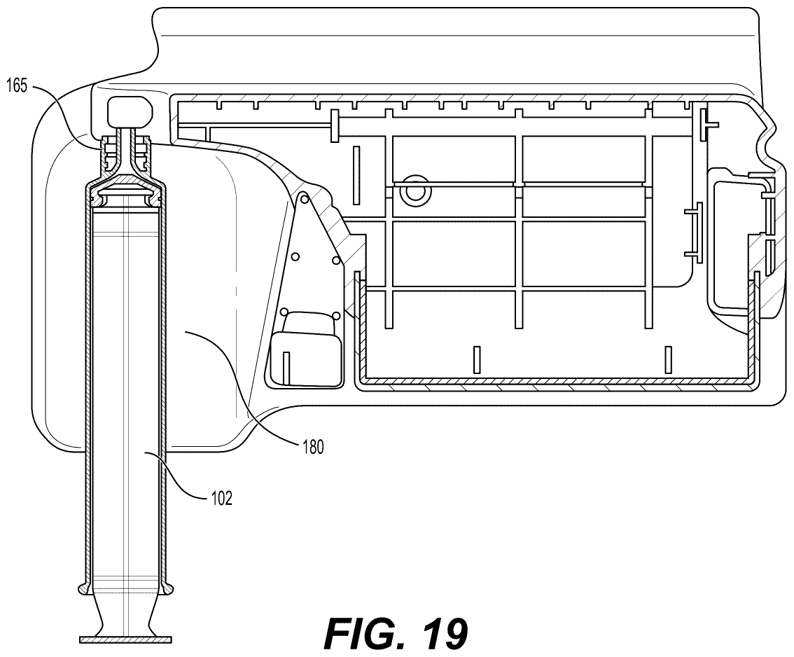

Still referring to FIG. 16, and referring also to FIGS. 17-19, after a process of mounting flow sensor 160 to base 180, a user may connect medical device 102 (e.g., a syringe, etc.) to fluid injection port 165. Fluid injection port 165 may include a needless connector or a needle-free piston (e.g., a BD SmartSite® needle-free connector, etc.) that compresses or decompresses when medical device 102 (e.g., a syringe, etc.) is either connected to or disconnected from fluid injection port 165. For example, fluid injection port 165 may include a threaded connector (e.g., an externally threaded connector, a luer lock thread or groove, etc.), and medical device 102 (e.g., a syringe, etc.) may include a complementary threaded connector (e.g., a luer tip surrounded by an internally threaded connector, a complementary luer lock thread or groove, etc.) configured to complementarily mate with the threaded connector of fluid injection port 165. As an example, fluid injection port 165 may include a needless connector as described in U.S. patent application Ser. No. 18/630,427, filed on Apr. 9, 2024, which is incorporated herein by reference in its entirety. A resulting compression between flow sensor 160 and base 180 may serve as a measurable indicator of the interaction between flow sensor 160 medical device 102. For example, when medical device 102 is connected to or disconnected from fluid injection port 165 of flow sensor 160, the two attachment or latch points L1 and L2 including the interface between first arced flange 1708 of flow sensor housing 1002 and second arced flange 1760 of base housing 1004 at the pivot point at the first side 1752a at the second end 1750b of base housing 1004 of base 180 and the retaining latch/snap interface with beam 1704 and beam tab 1706 at the first side 1752a at the first end 1750a of base housing 1004 of base 180 may transfer the applied force to base housing 1004 of base 180.

Still referring to FIGS. 16-19, to connect medical device 102 (e.g., a syringe, etc.) to fluid injection port 165, a user may hold base 180 with one hand and apply force with another hand to initially press medical device 102 (e.g., a syringe, etc.) into fluid injection port 165. For example, a luer tip of medical device 102 may be received within fluid injection port 165, which may compress a bellows or piston 198 (FIG. 15) therein and/or apply a compression force via an inner wall of fluid injection port 165. As an example, a force used to compress the bellows or piston 198 may be approximately 12N. The compression force resulting from the initial pressing of medical device 102 into fluid injection port may be transferred from flow sensor 160 to base 180, and the least one force sensor 190 may measure the transferred compression force to generate a force signal corresponding to or representative of the compression force. For example, flow sensor housing 1002 may react against the compression force and transfer the compression force to base housing 1004.

After this initial insertion of the luer tip of medical device 102 (e.g., a syringe, etc.) into fluid injection port 165, the user may rotate medical device 102 (e.g., by 180 degrees, etc.) to achieve a full insertion depth. For example, the threaded connector of fluid injection port 165 and the complementary threaded connector of medical device 102 may mate in a complementary manner enabling medical device 102 to continue to the full insertion depth within fluid injection portion 165 and secure medical device 102 to fluid injection port 165. As an example, during the rotation or mating, torque is applied to the threaded connector of fluid injection port 165 and transferred to base 180. The torsional force resulting from the rotation of medical device 102 when medical device 102 is within fluid injection port 165 may be transferred from flow sensor 160 to base 180, and the at least one force sensor 190 may measure the transferred torsional force to to generate a force signal corresponding to or indicative of the torsional force.

Accordingly, as a user performs these actions to connect medical device 102 to fluid injection port 165, a force exerted on flow sensor 160 via medical device 102 may be transferred to the at least one force sensor 190 in base 180 and used to generate a force signal corresponding to or indicative of the compressive and/or torsional force used to connect medical device 102 to fluid injection port 165. Similarly, as a user performs these actions in reverse to disconnect medical device 102 from fluid injection port 165, a force exerted on flow sensor 160 via medical device 102 may be transferred to the at least one force sensor 190 in base 180 and used to generate a force signal corresponding to or indicative of a tension and/or opposite torsional force used to disconnect medical device 102 from fluid injection port 165.

The one or more processors 204 of base 180 may process force signals corresponding to the measured forces associated with connecting or disconnecting medical device 102 to flow sensor 160 to establish a baseline measurement as a signature pattern for a connection or a disconnection of medical device 102 to flow sensor 160, and the baseline force signature may be used to compare actual syringe connection or disconnection events to determine syringe connection state. In some implementations, analyzing the at least one force signal includes providing the at least one force signal to a machine-learning model trained to identify medical device connection events. For example, the one or more processors 204 of base 180 and/or remote computing system 110 may analyze the at least one force signal to determine the medical device connection state by providing data associated with the at least one force signal as input to the machine learning model trained to determine the medical device state and receiving as output from the machine learning model the medical device connections state.

Referring now to FIGS. 20A-20C, the at least one force sensor 190 may include a first force sensor configured to generate a first force signal indicative of the at least one force applied to the base housing and a second force sensor configured to generate a second force signal indicative of the at least one force applied to the base housing, and the at least one force signal may include a difference between the first force signal and the second force signal that is indicative of at least one of the following: a tension force or a compression force applied to base housing 1004, a torsional force applied to base housing 1004, or any combination thereof.

In some implementations, and referring specifically to FIG. 20A, the first force sensor and the second force sensor may be mounted (e.g., on PCB 192, etc.) in-line with each other and parallel to a longitudinal axis of the fluid injection port, and the difference between the first force signal and the second force signal may be indicative of the tension force or the compression force applied to the base housing. For example, the first force sensor and the second force sensor may be mounted (e.g., on PCB 192, etc.) in-line with an expected compression force that is expected to result from an initial pressing of medical device 102 into fluid injection port 165. In this way, a difference between the first force signal and the second force signal may be indicative of a tension force or a compression force applied to base housing 1004.

In some implementations, and referring to FIG. 20B, the first force sensor and the second force sensor may be mounted (e.g., on PCB 192, etc.) in-line with each other and perpendicular to a longitudinal axis of the fluid injection port, and the difference between the first force signal and the second force signal may be indicative of the torsional force applied to the base housing. For example, the first force sensor and the second force sensor may be mounted (e.g., on PCB 192, etc.) in-line with each other and perpendicular to the expected compression force that is expected to result from the initial pressing of medical device 102 into fluid injection port 165 to capture a torsional force applied to base housing 1004 during rotation of medical device 102 within fluid injection port 165.

In some implementations, and still referring to FIG. 20B, the at least one force sensor 190 may further include a third force sensor configured to generate a third force signal indicative of the at least one force applied to the base housing and a fourth force sensor configured to generate a fourth force signal indicative of the at least one force applied to the base housing. The third force sensor and the fourth force sensor may be mounted in-line with each other and perpendicular to a longitudinal axis of the fluid injection port and parallel to the first force sensor and the second force sensor mounted in-line with each other, and the at least one force signal may include a difference between the first force signal or the second force signal and the third force signal or the fourth force signal that is indicative of at least one of the following: a tension force or a compression force applied to base housing 1004, a torsional force applied to the base housing, or any combination thereof. For example, a difference between force signals of force sensors mounted in-line with each other and parallel to the longitudinal axis of fluid injection port 165 may capture a tension force or a compression force applied to base housing 1004 during initial insertion of medical device 102 within fluid injection port 165, and a difference between force signals of force sensors mounted in-line with each other and perpendicular to the longitudinal axis of fluid injection port 165 may capture torsional force applied to base housing 1004 during rotation of medical device 102 within fluid injection port 165.

In some implementations, and referring to FIG. 20C, the first force sensor and the second force sensor may be mounted (e.g., on PCB 192, etc.) offset from each other along each of a longitudinal axis of the fluid injection port and a lateral axis of the fluid injection port, and the difference between the first force signal and the second force signal may be indicative of the combination of the tension force or the compression force applied to the base housing and the torsional force applied to the base housing. For example, a difference between force signals of force sensors mounted offset from each other along each of a longitudinal axis of the fluid injection port and a lateral axis of the fluid injection port may capture each of the tension force or the compression force applied to base housing 1004 during initial insertion of medical device 102 within fluid injection port 165 and the torsional force applied to base housing 1004 during rotation of medical device 102 within fluid injection port 165.

In some non-limiting embodiments or aspects, the one or more processors 204 of base 180 may provide an indication associated with a connection state of flow sensor 160 and base 180 and/or a connection state of medical device 102 to fluid injection port 165 of flow sensor 160. The indication may be a human perceivable output indicating the connection state (e.g., connected, disconnected, etc.) of flow sensor 160 and base 180 and/or the connection state (e.g., connected, disconnected, etc.) of medical device 102 to fluid injection port 165 of flow sensor 160 (e.g., provided via a speaker and/or a display of a reusable base of the flow sensor system in U.S. Patent Application Publication No. 2021/0231471, which is incorporated herein by reference in its entirety etc.). In some implementations, providing the indication may include storing a value in a location of a storage device for subsequent retrieval (e.g., in a memory base 180, in remote computing device 110, in a database etc.), transmitting a value directly to remote computing device 110 via at least one wired or wireless communication medium, transmitting or storing a reference to a value, and the like. Providing the indication may additionally or alternatively include encoding, decoding, encrypting, decrypting, validating, verifying, and the like via a hardware element.

In some non-limiting embodiments or aspects, the one or more processors 204 of base 180 and/or remote computing device 110 may provide the indication associated with a connection state of flow sensor 160 and base 180 and/or a connection state of medical device 102 to fluid injection port 165 of flow sensor 160 in association with patient data associated with a patient, procedure data associated with a patient procedure associated with the patient, caregiver data associated with a caregiver (e.g., a nurse, a doctor, etc.), any combination thereof, or the like. Patient data associated with a patient may include a patient identifier associated with the patient (e.g., a unique patient identifier, etc.), patient demographics (e.g., a name, an age, a sex, a weight, a height, a birthdate, an address, etc.), a list of medication allergies associated with the patient, a list of medication doses delivered, being delivered, and/or pending for delivery to the patient, any combination thereof, or the like. Procedure data may include a procedure identifier associated with the procedure (e.g., a unique procedure identifier, etc.), one or more medical devices associated with the procedure, a name of the procedure, a state of the procedure (e.g., scheduled for a future date and time, currently being performed, previously performed a previous date and time, etc.), a caregiver associated with the procedure, a patient associated with the procedure, any combination thereof, or the like. Caregiver data may include a caregiver identifier associated with the caregiver (e.g., a unique caregiver identifier, etc.), a name of the caregiver, any combination thereof, or the like.

In some non-limiting embodiments or aspects, the one or more processors 204 of base 180 may automatically control flow sensor system 150 to perform one or more operations in response to determining a connection state of flow sensor 160 and base 180 and/or a connection state of medical device 102 to fluid injection port 165 of flow sensor 160. For example, in response to determining that medical device 102 is connected to fluid injection port 165, the one or more processors 204 of base 180 may automatically control a short-range wireless communication device of base 180 to read short range wireless communication tag 104 on medical device 180 (e.g., to read a device identifier or other information associated with medical device 102 that is stored in short range wireless communication tag 104, etc.).

Accordingly, non-limiting embodiments or aspects of the present disclosure may provide a syringe force detection mechanism for a flow sensor system that enables the flow sensor system to automatically detect and confirm a connection or a disconnection of a syringe to the system without requiring any additional interaction from a user (e.g., the user need not to respond to prompts, press buttons, or manipulate levers to verify the system's current state, etc.).

Aspects described include artificial intelligence or other operations whereby the system processes inputs and generates outputs with apparent intelligence. The artificial intelligence may be implemented in whole or in part by a model. A model may be implemented as a machine learning model. The learning may be supervised, unsupervised, reinforced, or a hybrid learning whereby multiple learning techniques are employed to generate the model. The learning may be performed as part of training. Training the model may include obtaining a set of training data and adjusting characteristics of the model to obtain a desired model output. For example, three characteristics may be associated with a desired item location. In such instance, the training may include receiving the three characteristics as inputs to the model and adjusting the characteristics of the model such that for each set of three characteristics, the output device state matches the desired device state associated with the historical data.

In some implementations, the training may be dynamic. For example, the system may update the model using a set of events. The detectable properties from the events may be used to adjust the model.

The model may be an equation, artificial neural network, recurrent neural network, convolutional neural network, decision tree, or other machine-readable artificial intelligence structure. The characteristics of the structure available for adjusting during training may vary based on the model selected. For example, if a neural network is the selected model, characteristics may include input elements, network layers, node density, node activation thresholds, weights between nodes, input or output value weights, or the like. If the model is implemented as an equation (e.g., regression), the characteristics may include weights for the input parameters, thresholds, or limits for evaluating an output value, or criterion for selecting from a set of equations.

Once a model is trained, retraining may be included to refine or update the model to reflect additional data or specific operational conditions. The retraining may be based on one or more signals detected by a device described herein or as part of a method described herein. Upon detection of the designated signals, the system may activate a training process to adjust the model as described.

Further examples of machine learning and modeling features which may be included in the embodiments discussed above are described in “A survey of machine learning for big data processing” by Qiu et al. in EURASIP Journal on Advances in Signal Processing (2016) which is hereby incorporated by reference in its entirety.

Although embodiments have been described in detail for the purpose of illustration, it is to be understood that such detail is solely for that purpose and that the disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect.

What is claimed is:

1. A flow sensor system, comprising:
a flow sensor including:
a flow sensor housing;
a flow tube including a fluid inlet at a first end of the flow tube, a fluid outlet at a second end of the flow tube opposite the first end of the flow tube, and a fluid injection port between the first end and the second end of the flow tube; and
at least one sensor configured to characterize at least one attribute of a fluid in the flow tube; and
a base configured to connect to the flow sensor, wherein the base includes:
a base housing;
at least one force sensor configured to generate at least one force signal indicative of at least one force applied to the base housing; and
at least one processor configured to determine, based on the at least one force signal, a connection of a medical device to the fluid injection port of the flow sensor or a disconnection of the medical device from the fluid injection port of the flow sensor.

2. The flow sensor system of claim 1, wherein the at least one processor is further configured to determine, based on the at least one force signal, a connection of the flow sensor to the base or a disconnection of the flow sensor from the base.

3. The flow sensor system of claim 1, wherein the at least one force sensor includes a first force sensor configured to generate a first force signal indicative of the at least one force applied to the base housing and a second force sensor configured to generate a second force signal indicative of the at least one force applied to the base housing, and wherein the at least one force signal includes a difference between the first force signal and the second force signal that is indicative of at least one of the following: a tension force or a compression force applied to the base housing, a torsional force applied to the base housing, or any combination thereof.

4. The flow sensor system of claim 3, wherein the first force sensor and the second force sensor are mounted in-line with each other and parallel to a longitudinal axis of the fluid injection port, and wherein the difference between the first force signal and the second force signal is indicative of the tension force or the compression force applied to the base housing.

5. The flow sensor system of claim 3, wherein the first force sensor and the second force sensor are mounted in-line with each other and perpendicular to a longitudinal axis of the fluid injection port, and wherein the difference between the first force signal and the second force signal is indicative of the torsional force applied to the base housing.

6. The flow sensor system of claim 5, wherein the at least one force sensor further includes a third force sensor configured to generate a third force signal indicative of the at least one force applied to the base housing and a fourth force sensor configured to generate a fourth force signal indicative of the at least one force applied to the base housing, wherein the third force sensor and the fourth force sensor are mounted in-line with each other and perpendicular to a longitudinal axis of the fluid injection port and parallel to the first force sensor and the second force sensor mounted in-line with each other, and wherein the at least one force signal includes a difference between the first force signal or the second force signal and the third force signal or the fourth force signal that is indicative of at least one of the following: a tension force or a compression force applied to the base housing, a torsional force applied to the base housing, or any combination thereof.

7. The flow sensor system of claim 3, wherein the first force sensor and the second force sensor are offset from each other along each of a longitudinal axis of the fluid injection port and a lateral axis of the fluid injection port, and wherein the difference between the first force signal and the second force signal is indicative of the combination of the tension force or the compression force applied to the base housing and the torsional force applied to the base housing.

8. The flow sensor system of claim 1, wherein the base further includes:
a printed circuit board (PCB), wherein the at least one force sensor is mounted on the PCB.

9. The flow sensor system of claim 8, wherein the at least one force sensor is in direct physical contact with an inner surface of base housing.

10. The flow sensor system of claim 8, wherein the base further includes:
at least one actuator arm in physical contact with the at least one force sensor and an inner surface of the base housing, wherein the at least one actuator arm is configured to transfer at least a portion of the at least one force applied to the base housing to the at least one force sensor.

11. The flow sensor system of claim 1, wherein the flow sensor housing extends between a first end and a second end opposite the first end, wherein the fluid injection port extends from the flow sensor housing at the second end of the flow sensor housing, wherein the flow sensor housing includes a first arced flange that at least partially surrounds the fluid injection port,
wherein the base housing extends between a first end and a second end opposite the first end and between a first side and a second side opposite the first side, wherein the first side of the base housing includes an opening that extends between the first end and the second end opposite the first end, wherein the opening is configured to receive the flow sensor housing,
wherein the opening extends from the first side at the second end of the base housing toward the second side of the base housing as an at least partially open bore, wherein the at least partially open bore is partially surrounded by a second arced flange, wherein the second arced flange is configured to receive and partially surround the fluid injection port with the first arced flange of the flow sensor housing between the second arced flange of the base housing and the second side of the base housing, and
wherein the at least one force sensor is configured to receive the at least one force applied to the base housing via the second arced flange of the base housing to generate the at least one force signal indicative of the at least one force applied to the base housing.

12. The flow sensor system of claim 11, wherein the first arced flange of the flow sensor housing includes a first face that faces the flow sensor housing and a second face opposite the first face, wherein the second arced flange of the base housing includes a first face that faces the first side of the base housing and away from the second side of the base housing and a second face opposite the first face that faces toward the second side of the base housing, wherein, when the fluid injection port is received within and partially surrounded by the second arced flange of base housing, the first arced flange of the flow sensor housing is rotatable with respect to the second arced flange of the base housing to bring the first face of the first arced flange flow of the flow sensor housing into contact with the first face of the second arced flange of the base housing, and
wherein the at least one force sensor is configured to receive the at least one force applied to the base housing via the first face of the second arced flange of the base housing to generate the at least one force signal indicative of the at least one force applied to the base housing.

13. The flow sensor system of claim 1, wherein the flow sensor further includes a flow sensor electrical contact in electrical communication with the at least one sensor, wherein the base further includes a base electrical contact in electrical communication with the one or more processors, and wherein the flow sensor electrical contact is in electrical communication with the base electrical contact when the flow sensor is connected to the base.

14. The flow sensor system of claim 1, wherein the fluid injection port includes a threaded connector, and wherein the medical device includes a syringe including a complementary threaded connector configured to complementarily mate with the threaded connector of the fluid injection port.

15. A base for a flow sensor, comprising:
a base housing;
at least one force sensor configured to generate at least one force signal indicative of at least one force applied to the base housing; and
at least one processor configured to determine, based on the at least one force signal, a connection of a medical device to a fluid injection port of a flow sensor connected to the base or a disconnection of the medical device from the fluid injection port of the flow sensor connected to the base.

16. The base of claim 15, wherein the at least one processor is further configured to determine, based on the at least one force signal, a connection of the flow sensor to the base or a disconnection of the flow sensor from the base.

17. The base of claim 15, wherein the at least one force sensor includes a first force sensor configured to generate a first force signal indicative of the at least one force applied to the base housing and a second force sensor configured to generate a second force signal indicative of the at least one force applied to the base housing, and wherein the at least one force signal includes a difference between the first force signal and the second force signal that is indicative of at least one of the following: a tension force or a compression force applied to the base housing, a torsional force applied to the base housing, or any combination thereof.

18. The base of claim 15, wherein the base further includes:
a printed circuit board (PCB), wherein the at least one force sensor is mounted on the PCB.

19. The base of claim 18, wherein the base further includes:
at least one actuator arm in physical contact with the at least one force sensor and an inner surface of the base housing, wherein the at least one actuator arm is configured to transfer at least a portion of the at least one force applied to the base housing to the at least one force sensor.

20. A system, comprising:
a fluid injection port;
a housing connected to the fluid injection port;
at least one force sensor configured to generate at least one force signal indicative of at least one force applied to the housing; and
at least one processor configured to determine, based on the at least one force signal, a connection of a medical device to the fluid injection port or a disconnection of the medical device from the fluid injection port.
